(19) United States
(12) Patent Application Publication
(10) Pub. No.: US 2025/0270611 A1
(43) Pub. Date: Aug. 28, 2025

(54) MICROBIAL CHAIN ELONGATION SYSTEM WITH PRETREATMENT AND CARRIERS FOR THE RECOVERY OF CASEIN AND MEDIUM CHAIN FATTY ACIDS FROM ORGANIC BIOMASS

(71) Applicant: King Abdullah University of Science and Technology, Thuwal (SA)

(72) Inventors: Bin Bian, Thuwal (SA); Pascal Saikaly, Thuwal (SA); Krishna Katuri, Thuwal (SA); Muhammad Ali, Thuwal (SA); Jiajie Xu, Thuwal (SA)

(21) Appl. No.: 19/186,262

(22) Filed: Apr. 22, 2025

Related U.S. Application Data
(63) Continuation-in-part of application No. 18/044,522, filed on Mar. 8, 2023.
(60) Provisional application No. 63/076,266, filed on Sep. 9, 2020.

(51) Int. Cl. C12P 21/02; C12M 1/00; C12M 1/12; C12P 7/6409

(52) U.S. Cl. CPC C12P 21/02; C12M 25/18; C12M 29/24; C12P 7/6409

(57) ABSTRACT

Bioreactors for production and recovery of medium chain carboxylates from organic biomass are disclosed. Methods for improved production and recovery of medium chain carboxylates from organic biomass are also disclosed. The bioreactors can be used as a chain-elongation bioreactor, and a method of use thereof results in improved production and recovery of medium chain carboxylates from organic biomass. The bioreactor includes a shell defined by one or more walls and a length, and a plurality of porous hollow fiber membranes placed inside the reactor for continuous liquid-liquid extraction, as well as granular activated carbon (GAC) as biocarriers. The plurality of hollow fiber membranes is mounted such that a percentage of the length of the shell remains unoccupied by the plurality of porous hollow fiber membranes.

### CROSS REFERENCE TO RELATED APPLICATION

This application is a continuation-in-part of U.S. Ser. No. 18/044,522, filed Mar. 8, 2023, which claims the benefit of and priority to U.S. Provisional Application No. 63/076,266, filed Sep. 9, 2020, which is hereby incorporated herein by reference in its entirety.

### FIELD OF THE INVENTION

This invention relates to an improved process for production and recovery of medium chain carboxylates from organic biomass.

### BACKGROUND OF THE INVENTION

Carbon recovery from organic waste or wastewater reduces the cost of waste treatment and also increases recoverable chemical energy (Hao, et al., Water Res. 2019, 161, 74-77; Lu, et al., Nat. Sustain 2018, 1, 750-758). One of the biotechnologies that is of interest for renewable chemical production is the carbon chain elongation platform. Carbon chain elongation platform harnesses the potential of certain microbes in anaerobic fermentation biotechnology to generate medium-chain carboxylic acids (MCCAs, C6-C12) from short-chain carboxylic acids (SCCAs, C2-C5) and an electron donor (e.g., ethanol), which can be obtained through the hydrolysis of organic biomass (Angenent, et al., Environ. Sci. Technology 2016, 50, 2796-2810; Daly, et al., ACS Sustain Chem. Eng. 2020, 8, 13934-13944; Xu, et al., Joule 2018, 2, 280-295). The pathway of reverse β-oxidation is considered a thermodynamically favorable microbial synthesis pathway to produce MCCAS (Dellomonaco, et al., Nature 2011, 476, 355-359; González-Cabaleiro, et al., Energy Environ. Sci. 2013, 6, 378003789). MCCAs are valuable molecules and could be utilized for various industrial and agricultural applications, such as sustainable antimicrobials (Kim and Rhee, Appl. Environ. Microbiol. 2013, 79, 6552-6560), precursors for liquid biofuel production (Urban, et al., Energy. Environ. Sci. 2017, 10, 2231-2244), oleochemical production (Zhu, et al., Nat. Catal. 2020, 3, 64-74), and livestock feed additives for growth (Mills, et al., J. Dairy Sci. 2010, 93, 4262-4273). However, it is challenging to reach a high concentration of MCCAs in the microbial synthesis system due to the cellular toxicity of MCCAs (Zhu, et al., Nat. Catal. 2020, 3, 64-74). The uncharged carboxylic acids disrupt cell membrane. Further, these acids with longer carbon chains are more toxic due to the increased hydrophobicity of the carbon chain (Butkus, et al., Appl. Environ. Microbiol. 2011, 77, 363-366; Harroff, et al., Environ. Sci. Technol. 2017, 51, 9729-9738). Currently, an in-line extraction system for MCCAs is considered one of the best options for reducing cell membrane toxicity and end-product feedback inhibition, thus enabling high MCCAs production rates (Lambrecht, et al., Microb. Cell Fact. 2019, 18, 1-17; Michel-Savin, et al., Appl. Microbiol. Biotechnol. 1990, 33, 127-131; Roe, et al., Microbiology 2002, 148, 2215-2222).

Several technologies have been applied for MCCA in-line extraction directly from a fermentation broth, including electrodialysis cell (López-Garzón and Straathof, Biotechnol. Adv. 2014, 32, 873-904; Wang, et al., Bioresour. Technol. 2013, 147, 442-448), permeate membrane (Zhu, et al., ACS EST Eng. 2021, 1, 141-153), electrolysis unit (Carvajal-Arroyo, et al., Chem. Eng. J. 2020, 416, 127886), electrodialysis/phase separation cell (Xu, et al., Environ. Sci. Technol. 2021, 55, 634-644), and membrane-based liquid-liquid extraction (i.e., pertraction) (Gehring, et al., J. Chem. Technol. Biotechnol. 2020, 95, 3105-3116; Ge, et al., Environ. Sci. Technol. 2015, 49, 8012-8021; Saboe, et al., Green. Chem. 2018, 20, 179101804; Xu, et al., Chem. Commun. 2015, 51, 6847-6850). Pertraction for in-line extraction of MCCAs has been well studied and has already been applied in a pilot-scale system (CaproX) due to its low energy cost (mainly requiring electric power to pump the fermentation broth, hydrophobic solvent and pertraction solution) and selective extraction of the longest possible carbon chain of carboxylate (Angenent, et al., Bioresour. Technol. 2018, 247, 1085-1094). The driving force for MCCA pertraction is a pH gradient (˜5.0 to ˜9.0) to specifically extract undissociated carboxylic acids by diffusion through a forward and a backward membrane (Angenent, et al., Bioresour. Technol. 2018, 1085-1094). In accordance with previous pertraction mass transfer model study (Kucek, et al., Energy Environ. Sci. 2016b, 9, 3482-3494), an increase in the recycle flow rates of fermentation broth (0-225 m d⁻¹) led to an increase in the MCCA mass transfer rate. However, increasing the recycle flow rates of the extractant (e.g. hydrophobic solvent) or the pertraction solution (e.g. alkaline extraction solution) did not affect the overall mass transfer rates, indicating that mass transfer limitations were at the interface of the fermentation broth and the hydrophobic membrane contactor.

There is still a need for improved methods for production and recovery of medium chain carboxylates from organic biomass.

Accordingly, it is an object of the present invention to provide a system and method for organic biomass conversion into MCCA s system that permit more effective biological conversion of food waste as well as wastewater, and that reduces associated energy use and maintenance requirements.

### SUMMARY OF THE INVENTION

A chain-elongation carboxylate system disclosed is disclosed, which includes a bioreactor for production and recovery of medium chain carboxylates from organic biomass are disclosed. Methods for improved production and recovery of medium chain carboxylates from organic biomass are also disclosed. The bioreactors can be used as a chain-elongation bioreactor, and a method of use thereof results in improved production and recovery of medium chain carboxylates from organic biomass. The bioreactor includes: (i) a shell defined by one or more walls and a length, (ii) a submerged membrane, preferably a plurality of porous hollow fiber (HF) membranes placed inside the shell for continuous liquid-liquid extraction, and (iii) granular activated carbon (GAC), as biocarriers. The hollow fiber membrane/plurality of hollow fiber membranes is/are mounted such that a percentage of the length of the shell (e.g., between about 10% and about 70%) remains unoccupied by the hollow fiber membrane/plurality of porous hollow fiber membranes.

In some preferred embodiments, the size of GAC is from about 0.5 to about 1.5 mm. The GAC particles play a bi-functional role: 1) increase biomass concentration in the reactor; and 2) reduce membrane fouling; thus enhancing MCCA yield and lowering operational cost. The disclosed method, by addressing these two bottlenecks, result in improved product generation rates and yields.

The disclosed bioreactor and methods can be used in food waste treatment, high chemical oxygen demand (COD) wastewater treatment and bioenergy conversion. The disclosed bioreactor is used to improve methods for producing and sequestering carboxylates (e.g., C3 to C8 carboxylates or C6 to C12) from biomass using microorganisms.

Also disclosed is a three-stage system to valorize expired dairy and beverage waste mixture containing high concentrations of casein proteins for the separate recovery of casein proteins in the pretreatment stage (1st-stage), lactate as mediators in the fermentation stage (2nd-stage) and MCCAs as sustainable antibiotics, livestock feed additives, and precursors for liquid biofuels production in the chain elongation stage (3rd-stage) with plastic biocarriers preferably to enhance biomass retention.

In some forms, in the pretreatment stage (1st-stage), expired milk, yogurt, and fruit (total level of chemical oxygen demand (COD)>200 g L-1) are mixed at a ratio of 5:3.2:1.8 based on the actual percentage of each expired food in the total waste and are pretreated at 100° C. for 20 min to recover the casein proteins through centrifugation at 4000×g for 10 min. The centrifuged medium (CM, total COD level 50-80 g L-1) is then stored at 4° C. and used as a feedstock for the fermentation state (2nd-stage).

In the fermentation stage (2nd-stage), operation conditions (i.e., temperature and organic loading rate) are selected for enhanced lactate concentration and production rate.

In the chain elongation stage (3^{rd}-stage), plastic biocarriers are used to enhance biomass retention.

### BRIEF DESCRIPTION OF THE DRAWINGS

FIGS. 1A and 1B show the schematics of two pertraction strategies during Periods I to IX (Table 1): FIG. 1A shows a pertraction system using only internal hollow fiber membrane to extract MCCAs during Periods I to VI. Biogas recirculation was applied during Periods II to VI. FIG. 1B shows a pertraction system using internal and external hollow fiber membrane simultaneously to extract MCCAs during Periods VII to IX. Biogas recirculation was applied during Period VII. Broth recirculation was applied during Periods VIII to IX. HF: Hollow Fiber. Dash line represents the gas flow and solid line represents the liquid flow. FIG. 1C. shows hydraulic Retention Time (HRT) and loading rate during Periods I to IX. The bottom line represents the HRT and the orange line represents the loading rate. FIG. 1D is a graph showing Carboxylate mass transfer coefficient with abiotic synthetic broth during Stage A and B. C2: acetic acid; C4: n-butyric acid; C6: n-caproic acid; C8: n-caprylic acid. FIG. 1E is a graph showing solid concentrations during Periods I to IX. The top line represents the total solid concentration in the effluent. The bottom line represents the volatile solid concentration in the effluent.

FIGS. 2A-2C show the concentration of carboxylic acids in the bioreactor broth and biogas production during Periods I to IX. FIG. 2A is a stacked area chart for broth concentration of carboxylic acids (cumulative). FIG. 2B is a stacked area chart for a production rate of carboxylic acids including effluent, internal extraction and external extraction (cumulative). FIG. 2C is a line chart for biogas production rate (non-cumulative). FIG. 2D. shows ethanol concentration in the effluent during Periods I to IX.

FIG. 3 is a heatmap of relative OTU abundances of the nine microbiome samples collected during Periods I to IX. The top 20 OTUs with relative abundance ≥1% for one or more of the microbiome samples are listed. The OTUs are classified down to the lowest taxonomic level (o: order, f: family, g: genus) possible.

FIG. 4A shows tacked area charts for broth concentration of lactate and volatile fatty acids during Periods FeI to FeV in the fermentation reactor. FeI: 30° C., HRT 4 d, CM: TW 1:2; F-II: 35° C., HRT 4 d, CM: TW 1:2; F-III: 43° C., HRT 4 d, CM: TW 1:2; F-IV: 43° C., HRT 2 d, CM: TW 1:2; FeV: 43° C., HRT 2 d, CM without dilution. FIG. 4B is a Schematic of lactate-mediated MCCA production involving two-stage fermentation and chain elongation processes.

FIG. 5A. Line chart for the feeding medium composition into the chain elongation reactor during CE-I to CE-VI. The feed medium for Period CE-VI was collected from the first-stage fermentation reactor (around 100 L), and its actual concentration was measured before being fed into the chain elongation reactor. FIG. 5B. Stacked area chart for broth concentration of

ethanol, volatile fatty acids, and lactate in the chain elongation reactor during Periods CE-I to CE-VI. FIG. 5C. Stacked area chart for the production rate of butyrate and caproate during Periods CE-I to CE-VI in the chain elongation reactor, operated at a fixed HRT of 2.5 days.

FIG. 6 is a Heatmap of the relative abundance of the top 20 operational taxonomic units in the suspension of the fermentation reactor during Periods FeI to FeV. The taxa level shown on the left-hand side represents the genus level.

FIG. 7 shows co-occurrence networks between microbial OTUs in the fermentation reactor during Period FeI to FeV. Pink lines connecting nodes (with different sizes corresponding to their relative abundances) represent significantly positive correlations (p<0.01) between microbes, while blue lines indicate significantly negative correlation coefficients between two different microbes. Nodes with the same color form a module in the network and represent different OTUs that are highly interconnected and have only a few connections outside the group.

FIG. 8A. Heatmap of the relative abundance of the top 20 operational taxonomic units in the suspension of the chain elongation reactor operated with lactate only during Period CE-V and fermentation effluent in Period CE-VI. The taxa level shown on the left-hand side represents the genus level. FIG. 8B. Co-occurrence networks between microbial species in the chain elongation reactor during Periods CE-I to CE-VI. A, B, C, and D in the co-occurrence network represent the genera Methanobacterium, Methanobrevibacter, Methanothrix, and Methanothermobacter, respectively.

FIG. 9. Stacked area charts for the production rate of lactate and volatile fatty acids during Period F-I to F-V in the fermentation reactor.

FIG. 10. COD conversion efficiency and lactate selectivity calculated based on mM-C among all VFAs and lactate during Period F-I to F-V in the fermentation reactor.

FIG. 11. Biogas production from chain elongation reactor. Gas composition: H2: ˜81%, N2: ˜ 3%, and CH4: 16%.

FIG. 12. Heatmap of the relative abundance of the top 20 operational taxonomic units in the suspended biomass of the fermentation reactor before switching operation to continuous mode.

FIG. 13. Heatmap of the relative abundance of the top 20 operational taxonomic units in the inoculums used to seed the chain elongation reactor.

FIG. 14. Heatmap of the relative abundance of the top 20 operational taxonomic units in the suspension of the chain elongation reactor during Period CE-I and CE-IV. The taxa level shown on the left-hand side represents the genus level.

### DETAILED DESCRIPTION OF THE INVENTION

The chain-elongation carboxylate system disclosed herein is characterized in that it combines a fluidized bed bioreactor with membrane-based liquid-liquid extraction. It includes a bioreactor including active chain-elongation organisms; fluidized particles which function as support media to be attached by the chain-elongation organisms; and membranes including a submerged membrane module. The fluidized particles come into direct contact with the submerged membrane.

The disclosed bioreactor and methods of use thereof have three advantages: 1) reduce footprint of the extraction system, 2) increase biomass concentration in the reactor and 3) reduce membrane fouling.

### I. Definitions

The term “anaerobic fermentation” is used herein to mean a fermentation carried out under anaerobic conditions by eukaryotic or prokaryotic microorganisms, such as bacteria, fungi, algae or yeasts.

“Broth,” refers to the stream or media in a bioreactor containing a compound to be extracted. The compound can be a medium chain fatty acid (MCCA).

“Shell volume” refers to the volume of space enclosed by the shell of the bioreactor described herein.

### II. Bioreactors

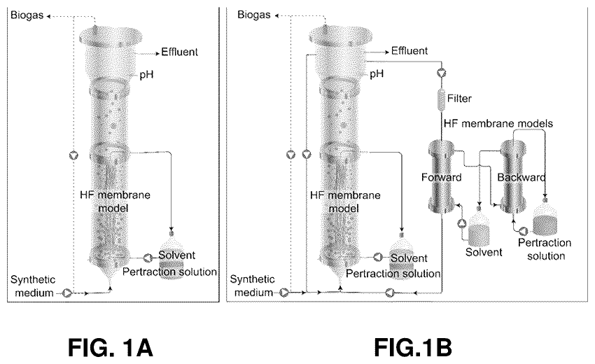

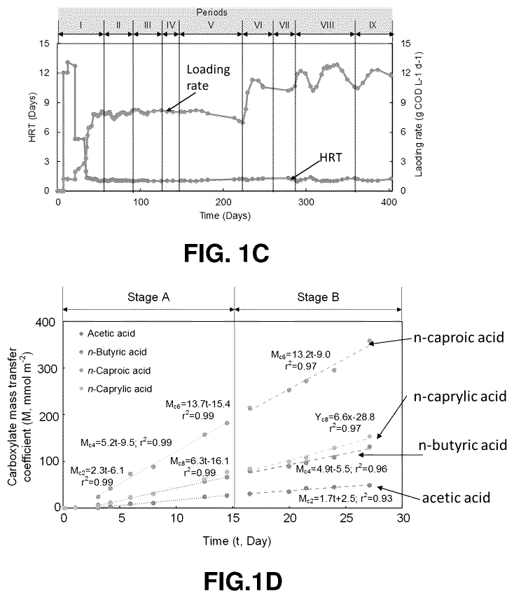

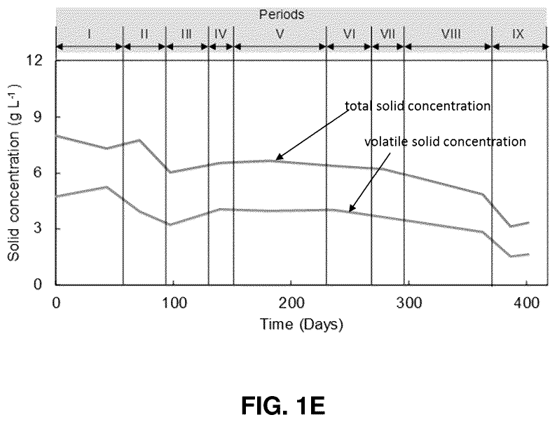

A bioreactor is disclosed herein (FIG. 1A), containing a shell and a submerged membrane module that is placed inside the shell for continuous liquid-liquid extraction. The shell is defined by one or more walls. The submerged membrane module contains a hollow fiber membrane, preferably, a plurality of hollow fiber membranes. Preferably, the hollow fibers in the plurality of hollow fiber membranes are porous. In some forms, the membrane module containing the plurality of hollow fiber membranes does not span the entire length of the shell, such that a length of between about 20% and about 50% of the length of the shell, remains unoccupied by the membrane module. In some forms, the inside of the shell can also contain granular activated carbon (GAC) particles as bio-carriers. GAC is used to control membrane fouling. GAC also possesses high surface area for colonization by microbes in the bioreactor. Thus, the GAC particles are expected to serve as bio-carriers for enhancing the colonization of chain-elongating thermophilic microbes in AnFMBR. The disclosed bioreactor preferably does not include a forward or backward hollow fiber membrane module (FIG. 1B).

### A. Shell

i. Materials

The shell of the bioreactors disclosed herein can be made from any material that provides sufficient strength and dimensional stability for carrying out the desired mass transfer operations. Examples of suitable materials include polypropylene, polyvinylidene fluoride, polyvinyl chloride, metals (such as silver, zinc, copper, aluminum, nickel, iron, titanium, and chromium), metal alloys of any of the preceding metals, ceramics, glass, borosilicate-tempered glass, steel (e.g., stainless steel, carbon steel, etc), plastics (e.g., epoxy resins, UV cured resins, thermosetting resins, etc), ceramics, composites, quartz, silicon, and combinations thereof.

ii. Shape

The shell can have a variety of different shapes, such as a cylinder, rectangle, square, pentagon, hexagon, octagon, etc. In some preferred forms, the shell has a cylindrical shape.

iii. Size

The design of the bioreactor is not limited by volumetric size, i.e., as determined by the dimensions of shell. For instance, the bioreactor can be an industrial scale reactor or a laboratory scale reactor. Laboratory scale reactors typically have shell volumes in the range of a few millimeters (e.g. 2 mL) to a few liters (e.g. 1 L, 2 L, 2.25 L, 3 L, or 5 L). In some forms, bioreactor volume is between 1 L and 5 L, such as 2.25 mL. In some forms, the bioreactor volume is between 0.1 m³ and 300 m³, such as 0.5 m³, from 0.45 m³ to 0.60 m³, 0.50 m³ to 0.60 m³, 1.0 m³, 2.0 m³, 3.0 m³, 4.0 m³, 5.0 m³, 10.0 m³, 20.0 m³, 25.0 m³, 50.0 m³, 75.0 m³, 100.0 m³, etc.

Where the shell is a cylinder, the cylinder can have an internal diameter between 3 cm and 10 cm, such as 5.5 cm. The cylinder can have height between 50 cm and 150 cm, such as 95 cm. In some forms, shell is a cylinder with a diameter of about 5.5 cm and a height of about 95 cm.

B. Hollow fiber membrane

The hollow fiber membranes for use in the disclosed bioreactors can be hydrophobic, hydrophilic, or a composite of both. Preferably, the hollow fibers are porous. In liquid/liquid extraction systems, low membrane mass transfer resistance can be obtained if the pores of the hollow fiber membranes contain a fluid in which the compound to be extracted is very soluble. Thus, a hydrophilic membrane or hydrophobic membrane can be used when the compound to be extracted is hydrophilic or hydrophobic, respectively.

i. Materials

The hollow fiber membranes disclosed herein, can be made from polymeric materials, non-polymeric materials, or a combination thereof. Materials for the hollow fiber membranes include, but are not limited, cellulose (e.g., regenerated cellulose), cellulose acetate, polysulfone, polyacrylonitrile, inorganic carbon, alumina, polypropylene, polyethylene, polyvinylidene fluoride, polytetrafluoroethylene, polyether sulfone, sulfonated polyether sulfone, and a combination thereof.

ii. Size

The hollow fiber membranes can have lengths that are suitable for a given mass transfer process. However, the lengths can be limited by the dimensions of the shell, the pumping costs that could be incurred by increasing the lengths of the hollow fiber membranes, or a combination thereof. Suitable lengths are between 5 cm and 50 cm, such as 44 cm; between 18 cm and 120 cm, between 18 cm and 185 cm, between 25 cm and 310 cm, between 60 and 110 cm, or a combination thereof. The lengths of the hollow fiber membranes can be, independent of the lengths of other hollow fiber membranes in the bioreactor, the same or different. In some forms, all the hollow fiber membranes have the same length. In some forms, the lengths of the hollow fiber membranes have a Gaussian distribution.

The hollow fiber membranes can have internal diameters that are suitable for a given mass transfer process. Suitable internal diameters can be between 0.1 mm and 10 mm, such as between 0.20 mm and 3 mm, between 0.5 mm and 3.5 mm, between 0.1 mm and 6 mm, between 0.5 mm and 1.5 mm. The internal diameters of the hollow fiber membranes can be, independent of the internal diameters of other hollow fiber membranes in the bioreactor, the same or different. In some forms, all the hollow fiber membranes have the same internal diameter. In some forms, the internal diameters of the hollow fiber membranes have a Gaussian distribution. The hollow fiber membranes can have wall thicknesses that are suitable for a given mass transfer process. Suitable wall thickness can be between 10 μm and 1 mm, such as between 30 μm and 0.5 mm. In some forms, the wall thickness is uniform over the length of the hollow fiber membranes. The wall thicknesses of the hollow fiber membranes can be, independent of the wall thicknesses of other hollow fiber membranes in the bioreactor, the same or different. In some forms, all the hollow fiber membranes have the same wall thickness. In some forms, the wall thicknesses of the hollow fiber membranes have a Gaussian distribution.

iii. Spacing/Density

Preferably, a plurality of hollow fiber membranes is assembled, i.e., potted, and mounted into the bioreactor's shell. Suitable materials for potting the hollow fiber membranes include polyepoxides (such as solvent-resistant polyepoxides), polyurethane, polypropylene, or a combination thereof. The hollow fiber membranes can be uniformly or non-uniformly distributed inside the shell. For instance, to obtain uniform spacing, the hollow fibers membranes can be woven into a fabric, potted, and mounted into the shell. In some forms, hollow fiber membranes are arranged in configurations such as cylindrical tube bundles, helically wound bundles, rectangular bed of fibers, or a combination thereof.

When a plurality of hollow fiber membranes is mounted into the shell, the packing density of the hollow fiber membranes preferably provides efficient fluidization of the hollow fiber membranes, which can give rise to high mass transfer rates. The packing density is the ratio of volume occupied by the hollow fiber membranes to the internal volume of the shell. In some forms, the packing density is at least 10% and less than 80%, such as 10%, 15%, 20%, 25%, 30%, 35%, 40%, 45%, 50%, 55%, 60%, 65%, and 70%.

The number of hollow fiber membranes can be selected, such that the fibers have a suitable interfacial area with the broth containing the compound to be extracted, a suitable volume utilization, or a combination thereof.

The plurality of hollow fiber membranes does not span the entire length of the shell, such that a percentage of the length of the shell remains unoccupied by the plurality of hollow fiber membranes. For example, one end of the plurality of hollow fiber membranes is mounted at a first end of the shell, while the other end of the plurality of hollow fiber membranes is mounted towards a second end of the shell, such that a length between about 10% and about 70%, between about 10% and about 60%, between about 10% and about 50%, between about 20% and about 50%, or between about 20% and about 30%, of the length of the shell, as measured from the second end, is left unoccupied by the plurality of hollow fiber membrane. For example, in some forms, one end of the plurality of hollow fiber membranes is mounted at a first end of the shell, while the other end of the plurality of hollow fiber membranes is mounted at the middle of the shell, i.e., about 50% the length of the shell, as measured from the second end, is left unoccupied by the plurality of hollow fiber membrane.

iv. Pore Sizes

The hollow fiber membranes can have pore sizes that are suitable for a given mass transfer process. Suitable pore sizes can be between 0.1 μm and 5 μm, such as between 0.1 μm and 0.2 μm, between 0.1 μm and 0.4 μm, between 0.1 μm and 0.65 μm, between 0.1 μm and 1 μm, between 0.2 μm and 0.4 μm, between 0.2 μm and 0.65 μm, between 0.4 μm and 0.65 μm, or a combination thereof. In some forms, the pore sizes uniform over the length of the hollow fiber membranes. The pore sizes of the hollow fiber membranes can be, independent of the pore sizes of other hollow fiber membranes in the bioreactor, the same or different. In some forms, all the hollow fiber membranes have the same porosity.

### C. Solvent Through Hollow Channel of Hollow Fiber

When the bioreactor is being used, one or more solvents flow through the hollow channel of a plurality of the hollow fiber membranes. Preferably, the hollow channel extends axially, i.e., along the length of the hollow fiber membrane, from one end to another end. In some forms, the one or more solvents are organic solvents. In some forms, the organic solvents are hydrophobic solvents. Suitable solvents include, but are not limited to, mineral oil solvent with tri-n-octylphosphine oxide (e.g., mineral oil solvent with 3% tri-n-octylphosphine oxide), N-methylpyrrolidone, methyl isobutyl ketone, xylene, n-butanol, 1,2-butanediol, and a combination thereof.

### D. Pertraction Solution

In liquid/liquid extraction systems, extraction of the compound that was extracted into the solvent flowing axially through the hollow fiber membranes often requires a second separation step. The second separation step can involve a solution, referred to herein as a pertraction solution. The pertraction solution contacts the solvent from the hollow fiber membranes and preferably remains phase-separated from the solvent. Preferably, the pertraction solution and solvent from the hollow fiber membranes are in direct contact, i.e., not separated by a membrane. During this phase-separated contact, the extracted compound(s) are stripped from the solvent from the hollow fiber membranes into the pertraction solution. In a scenario where the compound(s) extracted were carboxylic acids, the pertraction solution is an aqueous phase. Preferably, the pertraction solution is maintained at an alkaline pH, such as between 8 and 14, between 9 and 13, or between 9 and 11. Accordingly, the pertraction solution can contain a base (e.g., an inorganic base) such as sodium hydroxide or hydrogen carbonates, such as sodium hydrogen carbonate. In some forms, the pertraction solution can also contain small amounts of an acid (e.g. 0.2 M boric acid), but the overall pH is alkaline. For example, the pertraction solution can contain 0.2 M boric acid and 2 M sodium hydroxide solution. Preferably, the chemical components (e.g., bases) in the pertraction solution do not diffuse into the solvent from the hollow fibers, such that the extraction capability of the solvent remains stable.

### E. Materials to Sequester Microorganisms

The inside of the shell can also contain materials to sequester microorganisms. These materials are known as biocarriers. Biocarriers are generally inert, porous, and can sequester, retain, and enhance the number of microorganisms within their structure.

The biocarriers may be sand, granular activated carbon (GAC), glass, polystyrene beads, plastic materials of polypropylene, polyethylene, polyvinyl dichloride, polytetrafluoroethylene, latex, rubber, agarose, or other materials as commonly used in traditional fluidize bed reactors. In some forms, the plastic carrier is a plastic material. The size of GAC can be between about 0.5 mm and about 1.5 mm. The GAC of this size is effective in both colonizing organism and holding particulate matter, and prevent membrane clogging of MCCAs passing into organic solvent. Both the of submerged and back extraction membrane module are preferably hollow fiber membranes. The submerged hollow fiber membranes permit MCCAs in broth to go through membrane pore into organic solvent, but not the organisms, and broth, and prevents the organic solvent from flowing out through the pores of the hollow fiber membranes.

### III. Methods of Use

The disclosed bioreactor and methods can be used in food waste treatment, high COD wastewater treatment and bioenergy converting. Various sources of carbohydrate containing biomass can be used. For example, carbohydrate containing biomass can be municipal waste (food, yard, paper, organic fraction of source-sorted garbage, wood or biomass-based building materials, compost feedstocks), animal waste, agricultural residues (e.g., corn stover, corn fiber, wheat, barley, or rye straw, hay, silage, fruit or vegetable processing wastes), by-products of alternative energy processes (corn beer, sugar cane bagasse, butanol beer), wood wastes (e.g., saw mill, paper wastes, wooden pallets, building materials), biosolids wastes (waste activated sludge), animal hydrolysates (dead animals made soluble), waste from food production, such as cheese whey, yogurt production waste, beer production waste (including spent grain), or animal rendering waste. Other types of waste includes but is not limited to household waste such as fruits, vegetables, beverages and dairy products are typically the primary sources of household food waste. Waste carbon, including organic waste, wastewater, CO₂ and syngas converts into short carboxylates. Those short carboxylates can convert into medium chain carboxylates using the disclosed bioreactor and methods which use a microbial mixture packaged in fluidized particles.

The disclosed bioreactor is used to improve methods for producing and sequestering carboxylates (e.g., C3 to C8 carboxylates or C6 to C12 carboxylates) from biomass using microorganisms, via chain elongation.

Chain elongation is an open-culture biotechnological process which converts short chain fatty acids and an electron donor to medium chain fatty acids (M CFAs). Carbon chain elongation platform harnesses the potential of certain microbes in anaerobic fermentation biotechnology to generate medium-chain carboxylic acids (MCCAs, C6-C12) from short-chain carboxylic acids (SCCAs, C2-C5) and an electron donor (e.g., ethanol), which can be obtained through the hydrolysis of organic biomass. MCCAs are produced by certain bacteria in a strongly reduced anaerobic environment, via a metabolic pathway that has been recently reviewed by Spirito et al. [29]. The bacteria gain energy by combining the oxidation of an electron donor, i.e., lactic acid or ethanol, to acetyl-CoA with the reductive elongation of acetyl-CoA with acetic acid (C2), propionic acid (C3), butyric acid (C4), pentanoic acid (C5), or caproic acid (C6) generating a carboxylic acid with 2 additional carbons at each step.

Useful microorganisms include, but are not limited to those that effect chain elongation. Examples include Clostridium strains producing butyric acid and Megasphaera hexanoica producing caproic acid from the butyric acid. Clostridium kluyveri was the first isolated bacterium capable of producing caproic acid. Acidic pH are not favourable for growth of known ethanol chain elongators: the type-strain of C. kluyveri, strain DSM 555, has an optimum pH of 6.4, and grows in a pH range between 6 and 7.5. Another, more recent, isolate obtained from bovine rumen—strain 3231B—has been demonstrated to grow at pH as low as 4.88, although the optimal pH for growth of this strain also lies between pH 6.4 and 7.6. Biological production of hexanoic acid has been reported for a few strict anaerobic bacteria. Clostridium kluyveri produced hexanoic acid from ethanol, a mixture of cellulose and ethanol [5] and from ethanol and acetate. Strain BS-1, classified as a Clostridium cluster IV, produced hexanoic acid when cultured on galactitol. Megasphaera elsdenii produced a diverse mixture of carboxylic acids such as formic acid, acetic acid, propionic acid, butyric acid, pentanoic acid, and hexanoic acid from glucose and lactate and sucrose and butyrate. It is postulated that hexanoic acid is produced by two consecutive condensation reactions: the first is the formation of butyric acid from two acetyl-CoAs, and the second is the formation of hexanoic from one butyryl-CoA and one acetyl-CoA. The condensation reaction of two acetyl-CoAs to butyric acid has been well reported in Clostridium spp. such as Clostridium pasteurianum, C. acetobutylicum, and C. kluyveri . . . (reviewed in Jeon, et al., Biotechnology for Biofuels volume 9, 129 (2016) doi. 10.1186/s13068-016-0549-3.)

In some embodiments, the disclosed methods use mixed microbial culture (MMC). MMC use the synergy of bio-catalytic activities from different microorganisms to transform complex organic feedstock, such as by-products from food production and food waste. In the absence of oxygen, the feedstock can be converted into biogas through the established anaerobic digestion (A D) approach. (reviewed in Groof, et al., Molecules 2019, 24, 398; doi: 10.3390/molecules24030398).

Currently, anaerobic digestion is the most used bioprocess for the treatment of food waste with concomitant generation of biogas. However, to achieve a circular carbon economy, the organics in food waste should be converted to new chemicals with higher value than energy. The studies herein demonstrate the feasibility of medium-chain carboxylic acid (MCCA) production from expired dairy and beverage waste via a chain elongation platform mediated by lactate, in a two-stage process.

Thus in some embodiments, a three stage process is disclosed which includes a two-stage fermentation process and a pre-treatment step, as follows: a separate recovery of casein proteins in the pretreatment stage (1st-stage), lactate as mediators in the fermentation stage (2nd-stage) and MCCAs as sustainable antibiotics, livestock feed additives, and precursors for liquid biofuels production in the chain elongation stage (3rd-stage) with plastic biocarriers preferably to enhance biomass retention.

In some forms, in the pretreatment stage (1st-stage), expired milk, yogurt, and fruit (total level of chemical oxygen demand (COD)>200 g L-1) are mixed at a ratio of 5:3.2:1.8 based on the actual percentage of each expired food in the total waste and are pretreated at 100° C. for 20 min to recover the casein proteins through centrifugation at 4000×g for 10 min. The centrifuged medium (CM, total COD level 50-80 g L-1) is then stored at 4° C. and used as a feedstock for the fermentation state (2nd-stage).

The first stage of the two-stage fermentation process utilizes a fermentation bioreactor to transform the biomass, such as expired dairy and beverage waste, used to produce and sequester carboxylates (e.g., C3 to C8 carboxylates or C6 to C12 carboxylates) into a solution that contains lactate at a concentration ranging between 800 and 2000 mM C, between 800 and 1800 mM C, between 800 and 1600 mM C, between 800 and 1400 mM C, between 800 and 1200 mM C, or between 800 and 1000 mM C at 43° C.

In the second stage of the two-stage fermentation process, this lactate solution is then flowed into the chain elongation bioreactor as an inlet stream, where the lactate is used to produce carboxylates, such as caproate at a concentration ranging between 500 and 2000 mM C, between 500 and 1800 mM C, between 500 and 1600 mM C, between 500 and 1400 mM C, between 500 and 1200 mM C, or between 500 and 1000 mM C; and butyrate at a concentration ranging between 300 and 1200 mM C, between 300 and 1000 mM C, between 300 and 800 mM C, between 300 and 600 mM C, between 300 and 500 mM C, or between 300 and 400 mM C; via microbial chain elongation.

In some embodiments, the fermentation bioreactor of the first stage is initially inoculated with aerobic or anaerobic sludge, preferably anerobic sludge, from a bioreactor, such as a continuous stirred tank reactor (CSTR). Next, the fermentation bioreactor of the first stage undergoes acclimatization by being fed organic mass, such as expired milk, yogurt, and fruit juice, mixed at a ratio based on the actual percentage of each expired organic mass in the total organic mass and runs in batch operation. Other suitable methods for inoculation and acclimatization are well known in the art. After acclimatization, the fermentation bioreactor undergoes continuous operation, wherein the fermentation bioreactor is then operated in continuous mode without biomass recirculation or settling and fed with treated biomass feedstock. This treated biomass is heated between 90° C. and 110° C., between 95° C. and 105° C., between 98° C. and 102° C., or between 99° C. and 101° C., for 10 to 30 minutes, 15 to 25 minutes, 18 to 22 minutes, or 19 to 20 minutes. Preferably, the treated biomass is heated at 100° C. for 20 min. The biomass is also centrifuged between 3500×g and 4500×g, between 3700×g and 4300×g, or between 3900×g and 4100×g for 5 to 15 minutes, 7 to 13 minutes, or 9 to 11 minutes. Preferably, the biomass is centrifuged at 4000×g for 10 min to remove excessive protein content. The centrifuged medium is then stored at 4° C. and used as a feedstock for the fermentation reactor.

In some embodiments, the continuous mode operation step is conducted with a temperature between 25 and 55° C., between 25 and 50° C., between 25 and 45° C. or between 3° and 45° C.

In some embodiments, the continuous mode operation step is conducted with a Hydraulic Retention Time (HRT) between 1 and 6 d, between 1 and 5 d, between 1 and 4 d, between 1 and 3 d, or between 1 and 2 d.

In some embodiments, the continuous mode operation step is conducted with an organic loading rate between 1.0 and 40.0 g COD L⁻¹ d⁻¹, between 1.0 and 35.0 g COD L⁻¹ d⁻¹, between 1.0 and 30.0 g COD L⁻¹ d⁻¹, between 2.0 and 30.0 g COD L⁻¹ d⁻¹, between 3.0 and 30.0 g COD L⁻¹ d⁻¹, or between 4.0 and 30.0 g COD L⁻¹ d⁻¹.

In some embodiments, continuous operation of the first-stage fermentation bioreactor is divided into five periods to maintain conditions for enhancing lactate concentration and production rate. Conditions such as pH, temperature and organic loading rate influence the lactate concentration and production rate.

Period one of the operational parameters for the fermentation reactor operates between 25 and 50 days, between 25 and 45 day, between, 25 and 40 days, between 25 and 40 days, between 30 and 40 days, between 35 and 40 days, or between 37 and 39 days, at a temperature between 25° C. and 35° C., between 28° C. and 32° C., between 29° C. and 31° C., preferably at a temperature of 30° C., with a hydraulic retention time (HRT) between 2 and 6, between 3 and 5 d, and an organic loading rate between 4 and 6 g COD L⁻¹d⁻¹, between 4.5 and 5.5 g COD L⁻¹d⁻¹, between 4.7 and 5.3 g COD L⁻¹d⁻¹, or between 4.8 and 5.0 g COD L⁻¹d⁻¹, preferably an organic loading of 4.9 g COD L⁻¹d⁻¹.

Period two of the operational parameters for the fermentation reactor operates between 5 and 25 days, between 5 and 20 days, between 10 and 20 days, or between 14 and 16 days, at a temperature between 30° C. and 40° C., between 32° C. and 38° C., or between 34° C. and 36° C., preferably at a temperature of 35° C., with a hydraulic retention time (HRT) between 2 and 6, between 3 and 5 d, and an organic loading rate between 4 and 6 g COD L⁻¹d⁻¹, between 4.5 and 5.5 g COD L⁻¹d⁻¹, between 4.7 and 5.3 g COD L⁻¹d⁻¹, preferably and organic loading of 5.1 g COD L⁻¹d⁻¹.

Period three of the operational parameters for the fermentation reactor operates between 20 and 40 days, between 20 and 35 days, between 25 and 35 days, between 30 and 35 days, or between 30 and 32 days, at a temperature between 35° C. and 55° C., between 38° C. and 52° C., between 40° C. and 49° C., between 41° C. and 45° C., or between 42° C. and 44° C., preferably at a temperature of 43° C., with a hydraulic retention time (HRT) between 2 and 6, between 3 and 5 d, and an organic loading rate between 3.5 and 5.5 g COD L⁻¹d⁻¹, between 3.7 and 5.2 g COD L⁻¹d⁻¹, between 3.9 and 4.9 g COD L⁻¹d⁻¹, between 4.0 and 4.4 g COD L⁻¹d⁻¹, and between 4.1 and 4.3 g COD L⁻¹d⁻¹, preferably an organic loading rate of 4.2 g COD L⁻¹d⁻¹.

Period four of the operational parameters for the fermentation reactor operates between 40 and 60 days, between 40 and 55 days, between 40 and 50 days, between 45 and 50 days, or between 46 and 48 days, at a temperature between 35° C. and 55° C., between 38° C. and 52° C., between 40° C. and 49° C., between 41° C. and 45° C., or between 42° C. and 44° C., preferably at a temperature of 43° C., with a hydraulic retention time (HRT) between 1 and 6 d, between 1 and 5 d, between 1 and 4 d or between 1 and 3 d, and an organic loading rate between 7.5 and 9.5 g COD L⁻¹d⁻¹, between 7.7 and 9.2 g COD L⁻¹d⁻¹, between 7.9 and 8.9 g COD L⁻¹d⁻¹, between 8.1 and 8.7 g COD L⁻¹d⁻¹, between 8.3 and 8.5 g COD L⁻¹d⁻¹, preferably an organic loading rate of 8.4 g COD L⁻¹d⁻¹.

Period five of the operational parameters for the fermentation reactor operates for between 20 and 40 days, between 20 and 35 days, between 25 and 35 days, between 30 and 35 days, or between 30 and 32 days, at a temperature between 35° C. and 55° C., between 38° C. and 52° C., between 40° C. and 49° C., between 41° C. and 45° C., or between 42° C. and 44° C., preferably at a temperature of 43° C., with a hydraulic retention time (HRT) with a hydraulic retention time (HRT) between 1 and 6 d, between 1 and 5 d, between 1 and 4 d or between 1 and 3 d, and an organic loading rate between 20 and 35 g COD L⁻¹d⁻¹, between 22 and 33 g COD L⁻¹d⁻¹, between 24 and 31 g COD L⁻¹d⁻¹, between 26 and 29 g COD L⁻¹d⁻¹, 27 and 29 g COD L⁻¹d⁻¹, preferably an organic loading rate of 28.1 g COD L⁻¹d⁻¹.

The pH of the fermentation bioreactor is maintained between 3 and 7, between 4 and 6, between 5 and 5.5, or between 5.1 and 5.3. In some embodiments, this is done by periodically adding a suitable basic solution, such as sodium hydroxide solution (1 M). The agitation is fixed by a control panel between 50 and 150 rpm, between 50 and 140 rpm, between 50 and 130 rpm, between 50 and 120 rpm, between 50 and 110 rpm, between 60 and 110 rpm, between 70 and 110 rpm, between 80 and 110 rpm, or between 90 and 110 rpm.

Useful microorganisms include, but are not limited to those that promote lactate production, such as Lactobacillus, Lacticaseibacillus, Lactococcus, Enterococcus, Pediococcus, Leuconostoc, Streptococcus, Carnobacterium, Fructobacillus, Oenococcus, Sporolactobacillus, Tetragenococcus, Vagococcus, Aerococcus and Weissella. Preferably, the microorganisms of the fermentation bioreactor are Lactobacillus and Lacticaseibacillus.

### A. Types of Flow and Flow Rates

In use, a broth flows on the shell side of the bioreactor, and is in contact with the external surfaces of the hollow fiber membranes. Further, as solvent flows axially through the hollow channel of a plurality of the hollow fiber membranes. The solvent and the broth are separated by an interface formed by the walls of the hollow fiber membranes. As the broth flows over the hollow fiber membranes, a compound to be extracted from the broth diffuses across the membrane into the solvent.

The broth can be produced when an inlet stream flows into the bioreactor, and microorganisms metabolize one or more components in the inlet stream to produce a compound to be extracted. The inlet stream can also be the broth that already contains the compound to be extracted. As the inlet stream flows through the bioreactor and contacts the plurality of hollow fibers, (i) microorganisms (when present) in the bioreactor convert a component of the inlet stream into a product and/or a chemical compound to be extracted, and/or (ii) a compound is extracted from the inlet stream across the plurality of hollow fiber membranes. Thus, within the bioreactor, the inlet stream is generally a combination of some or all of its initial components and/or products. However, for simplicity, the inlet stream modified within the bioreactor, as described herein, is referred to as the shell side stream. Preferably, (i) the inlet fluid flows continuously into the bioreactor; (ii) the compound is extracted continuously; (iii) the solvent flows continuously through the hollow channels of the hollow fiber membranes; (iv) an outlet stream (for example an effluent) continuously exits the bioreactor; or a combination of (i), (ii), (iii), and (iv), such as (i)-(iv).

In some forms, the broth and the solvent flowing axially in one or more hollow fiber membranes flow in a co-current pattern, a counter-current pattern, a cross-current pattern, or a combination thereof. In some forms, the broth and the solvent flowing axially in one or more hollow fiber membranes flow in a co-current pattern. In some forms, the broth and the solvent flowing axially in one or more hollow fiber membranes flow in a counter-current pattern. In some forms, the broth and the solvent flowing axially in one or more hollow fiber membranes flow in a cross-current pattern.

In some forms, biogas produced in the bioreactor is recirculated into the bioreactor. In some forms, broth is recirculated into the bioreactor.

Generally, a shell side stream flows at a flow rate such that a solvent flowing axially through a plurality of hollow fiber membranes can extract a compound from the shell side stream. In some forms (such as for a 2-L bioreactor) the inlet flow rate is about 2 L/day or the hydraulic retention time is about one day.

### B. PH and Temperature

Further, operational temperature and pH conditions are conducive for compound extraction. In some forms, the inlet stream is provided at a temperature between 4° C. and 35° C., such as 4° C. In some forms, the temperature within the bioreactor is between 28° C. and 35° C. In some forms, pH of the bioreactor is maintained between 5 and 6, such as 5.5.

In some forms, (i) the pH of the bioreactor broth was maintained at 5.5; (ii) the hydraulic retention time was about one day; (iii) and biogas was recirculated every 2 hrs for 5 mins, at a rate of 150 mL/min. Optionally, the temperature of the bioreactor was maintained at about 32° C., such as 32±1° C.

The methods of use provide a process for extracting MCCA, produced by microorganisms in a fermentation reactor by anaerobic fermentation from fermentable biomass, preferably by of liquid-liquid type extraction. The process includes least the steps of bringing an extraction solvent into contact with a fermentation medium and separating the fermentative metabolites from the extraction solvent.

The disclosed bioreactor and methods of use can be further understood through the following enumerated paragraphs or embodiments.

1. A bioreactor containing:
a shell defined by one or more walls and a length, and
a plurality of hollow fiber membranes inside the shell,
wherein the plurality of porous hollow fiber membranes does not span the entire length of the shell.

2. The bioreactor of paragraph 1, wherein between about 10% and about 70%, between about 10% and about 60%, between about 10% and about 50%, between about 20% and about 50%, between about 20% and about 30%, or about 50% of the length of the shell remains unoccupied by the plurality of porous hollow fiber membranes.

3. The bioreactor of paragraph 1 or 2, wherein one end of the plurality of porous hollow fiber membranes is mounted at a first end of the shell and the other end of the plurality of porous hollow fiber membranes is mounted at a second portion of the shell.

4. The bioreactor of any one of paragraphs 1 to 3, wherein one end of the plurality of porous fiber membranes is mounted at a first end of the shell and the other end of the plurality of porous hollow fiber membranes is mounted at about the middle of the shell.

5. The bioreactor of any one of paragraphs 1 to 4, wherein the plurality of porous hollow fiber membranes contains polymeric materials, non-polymeric materials, or a combination thereof.

6. The bioreactor of any one of paragraphs 1 to 5, wherein hollow fiber membranes in the plurality of porous hollow fiber membranes contain cellulose (e.g., regenerated cellulose), cellulose acetate, polysulfone, polyacrylonitrile, inorganic carbon, alumina, polypropylene, polyethylene, polyvinylidene fluoride, polytetrafluoroethylene, polyether sulfone, sulfonated polyether sulfone, or a combination thereof.

7. The bioreactor of any one of paragraphs 1 to 6, wherein porous hollow fiber membranes in the plurality of porous hollow fiber membranes are potted at both ends with a material selected from polyepoxides (such as solvent-resistant polyepoxides), polyurethane, polypropylene, or a combination thereof.

8. The bioreactor of any one of paragraphs 1 to 7, wherein the plurality of porous hollow fiber membranes is configured as cylindrical tube bundles, helically wound bundles, rectangular bed of fibers, or a combination thereof.

9. The bioreactor of any one of paragraphs 1 to 8, wherein the shell has a shape selected from a cylinder, rectangle, square, pentagon, hexagon, or octagon.

10. The bioreactor of any one of paragraphs 1 to 9, wherein the shell contains a material selected from polypropylene, polyvinylidene fluoride, polyvinyl chloride, metals (such as silver, zinc, copper, aluminum, nickel, iron, titanium, and chromium), metal alloys of any of the preceding metals, ceramics, glass, borosilicate-tempered glass, steel (e.g., stainless steel, carbon steel, etc), plastics (e.g., epoxy resins, UV cured resins, thermosetting resins, etc), ceramics, composites, quartz, silicon, or a combination thereof.

11. The bioreactor of any one of paragraphs 1 to 10, wherein the bioreactor contains biocarriers in the shell volume.

12. The bioreactor of paragraph 11, wherein the biocarriers are selected from granular activated carbon, glass, polystyrene beads, plastic materials of polypropylene, polyethylene, polyvinyl dichloride, polytetrafluoroethylene, latex, rubber, agarose, or a combination thereof.

13. The bioreactor of any one of paragraphs 1 to 12, containing microorganisms.

14. The bioreactor of paragraph 13, wherein the microorganisms are sequestered on the biocarriers, within pore spaces of the biocarriers, or a combination thereof.

15. The bioreactor of paragraph 13 or 14, wherein the microorganisms include active chain-elongation organisms.

16. A method of extracting one or more compounds from a broth, the method involving:
contacting a shell side stream containing the broth with the plurality of porous hollow fiber membranes of the bioreactor of any one of claims 1 to 15.

17. The method of paragraph 16, wherein a solvent flows axially through the plurality of porous hollow fiber membranes.

18. The method of paragraph 17, wherein the shell side stream and solvent flowing axially through the plurality of porous hollow fiber membranes flow in a co-current pattern, a counter-current pattern, or a cross-current pattern, or a combination thereof.

19. The method of paragraph 17 or 18, wherein the shell side stream and the solvent flowing axially through the plurality of porous hollow fiber membranes flow in a co-current pattern.

20. The method of paragraph 18 or 19, wherein the solvent flowing axially through the plurality of porous hollow fiber membranes contains mineral oil solvent with tri-n-octylphosphine oxide (e.g., mineral oil solvent with 3% tri-n-octylphosphine oxide), N-methylpyrrolidone, methyl isobutyl ketone, xylene, n-butanol, 1,2-butanediol, or a combination thereof.

21. The method of any one of paragraphs 18 to 20, wherein the solvent flowing axially through the plurality of porous hollow fiber membranes contains mineral oil solvent with tri-n-octylphosphine oxide (e.g., mineral oil solvent with 3% tri-n-octylphosphine oxide).

22. The method of any one of paragraphs 17 to 21, the method involving:
contacting the solvent that flows axially through the plurality of porous hollow fiber membranes with a pertraction solution after the solvent exits the plurality of porous hollow fiber membranes.

23. The method of paragraph 22, wherein the pertraction solution has an alkaline pH, such as between 8 and 14, between 9 and 13, or between 9 and 11.

24. The method of paragraph 22 or 23, wherein the pertraction solution has a pH between 9 and 11.

25. The method of any one of paragraphs 16 to 24, wherein the bioreactor is maintained at a temperature between 28° C. and 35° C.

26. The method of any one of paragraphs 16 to 25, wherein the shell side stream containing the broth is maintained at a pH between 5 and 6, such as 5.5

27. The method of any one of paragraphs 16 to 26, the method involving:
recirculating biogas through the bioreactor.

28. The method of any one of paragraphs 16 to 27, wherein:
(i) the pH of the bioreactor broth is maintained at 5.5,
(ii) the bioreactor has a hydraulic retention time of about one day, and
(iii) biogas is recirculated every 2 hrs for 5 mins, at a rate of 150 mL/min.

29. The method of any one of paragraphs 16 to 28, wherein the one or more compounds are medium chain carboxylic acids.

### EXAMPLES

### Example 1

The objective of this work was to demonstrate the technical feasibility of utilizing a submerged (i.e., internal) hollow fiber membrane model in the bioreactor for MCCA extraction.

### Materials and Methods

### Substrate and Inoculum

Synthetic basal medium for the biotic experiments was prepared according to a previous study (Kucek, et al., Energy Environ. Sci. 2016b, 9, 3482-3494) with the following exceptions: yeast extract (1 g L-1) and sodium bicarbonate (1 g L-1). Two different concentration ratios of acetate to ethanol were applied during the nine periods to maintain sufficient ethanol in the influent (Table 1).

TABLE 1
Experimental approach and operating conditions for submerged
pertraction bioreactor during Periods I to IX.

| | Date | | | Influent |
|---|---|---|---|---|
| Periods | (Days) | Anti-fouling strategy | Pertraction type | (Ethanol:Acetate)^{a} |
| I | 0-50 | No | Internal | 50:25 |
| II | 51-83 | Biogas recirculation every 4 hrs | Internal | 50:25 |
| | | for 1 min at 40 mL min⁻¹ | | |
| III | 84-132 | Biogas recirculation every 6 hrs | Internal | 50:25 |
| | | for 30 min at 20 mL min⁻¹ | | |
| IV | 133-149 | Biogas recirculation every 6 hrs | Internal | 50:25 |
| | | for 30 min at 80 mL min⁻¹ | | |
| V | 150-223 | Biogas recirculation every 2 hrs | Internal | 50:25 |
| | | for 5 min at 150 mL min⁻¹ | | |
| VI | 224-248 | Biogas recirculation every 2 hrs | Internal | 100:25 |
| | | for 5 min at 150 mL min⁻¹ | | |
| VII | 249-282 | Biogas recirculation every 2 hrs | Internal + external | 100:25 |
| | | for 5 min at 150 mL min⁻¹ | | |
| VIII | 283-374 | Broth recirculation at flow rate | Internal + external | 100:25 |
| | | of 300 mL min⁻¹ | | |
| IX | 375-402 | Broth recirculation at flow rate | Internal + external | 100:25 |
| | | of 1600 mL min⁻¹ | | |

^{a}The ratios are mol:mol.

The pH of the medium was adjusted to 5.50 with 4 M of sodium hydroxide. The synthetic 5 broth was prepared with 3 g L-1 of Na2SO4, 20 mM of acetate, 20 mM of n-butyrate, 10 mM of n-caproate, and 1 mM of n-caproate for the abiotic pertraction experiments. The pH of the synthetic broth was set at 5.5.

The reactor was inoculated with a mixed biomass consisting of mangrove sediments, wastewater sludge, granular sludge and anaerobic digestion sludge to achieve high microbial diversity in the mixed inoculum. The mangrove sediment was collected from the King Abdullah Monument area (Thuwal, Saudi Arabia). The wastewater sludge was collected from the wastewater treatment plant at King Abdullah University of Science and Technology. The granular sludge and anaerobic digestion sludge were derived from a full-scale aerobic granular sludge reactor (Ali, et al., Water Res. 2020, 170, 115345) and lab-scale anaerobic digestion reactor (Cheng, et al., Environ. Int. 2019, 133, 105165). Each of the inoculum sources was washed three times in a basal medium, and 100 mL of each inoculum was added to the bioreactor.

### Bioreactor Construction and Pertraction

The up-flow bioreactor contained a cylinder with an internal diameter of 5.5 cm and height of 95 cm (FIG. 1A), and had a working volume of 2.25 L. The temperature of the bioreactor was maintained at 32±1° C. using a recirculating water bath (MP-5H, Hinotek, China). The bioreactor broth pH was maintained at 5.5±0.1 by an automatic pH controller (400 pH/ORP, Cole-Parmar, USA) and a dosing pump to add sodium hydroxide solution (2 M). The biogas was collected and recorded by a flow gas meter (TG05, Ritter, Germany). The synthetic medium was continuously fed to the bioreactor from a refrigerated container (4° C.) using a peristaltic pump, maintaining a hydraulic retention time (HRT) of ˜1 day (FIG. 1C). The effluent continuously exited the bioreactor using an overflow pipe fixed near the top of the bioreactor.

MCCAs were continuously extracted from the bioreactor with two types of in-line pertraction: internal and external hollow fiber membrane. For the internal hollow fiber membrane pertraction, 4 hollow fiber membranes (Cleanfil-S Membrane, Kolon Industries, South Korea) 44 cm long each were assembled as a single bundle using polyepoxides (Flow-mix, Devcon, USA). One end of the bundle was connected to the bottom port of the bioreactor. The other end of the hollow fiber bundle was connected to the middle port of the bioreactor. Mineral oil solvent (VWR, USA) with 3% tri-n-octylphosphine oxide (TOPO) (Alfa Aesar, USA) was used as the hydrophobic solvent and it was recycled at an up-flow rate of 1 mL min-1 (Cerampump, Fluid metering, USA) through the hollow fiber membranes from a two-phase reservoir in which 200 mL of the hydrophobic solvent and 250-300 ml of the alkaline pertraction solution were phase-separated (FIG. 1A). The alkaline extraction solution was initially buffered with 0.2 M boric acid and was maintained at a pH of 9-11 with manual addition of 2 M sodium hydroxide solution.

For external hollow fiber membrane pertraction, a forward and a backward membrane models with a contact area of 0.75 m2 (MD063CP2N, Microdyn, Germany) were applied which is similar to those used in a previous study (FIG. 1B) (Xu, et al., Joule 2018, 2, 280-295). The bioreactor broth was continuously circulated through the exterior space of the forward membrane model at a flow rate of 50 mL min-1. A 5 μm pore size filter (GS-6sed/5, Pentek, USA) was placed before the forward membrane model to prevent membrane fouling and was replaced every month. A constant hydrophobic solvent was circulated at a flow rate of 30 mL min-1 through the interior of the forward and backward hollow fiber membrane models. An alkaline pertraction solution (2.5 L) from a well-mixed reservoir was circulated at a flow rate of 40 mL min-1 through the exterior of the backward hollow fiber membrane model. This alkaline pertraction solution was similar to the one used for the internal hollow fiber membrane pertraction.

### Experimental Periods

To reduce membrane fouling, two operating strategies were adopted (Table 1): biogas recirculation (Periods II to VII) and broth recycle flow rate (Periods VIII to IX). During Period I (start-up phase), the bioreactor was operated for 50 days without any anti-fouling treatment. During Periods II to VII, successive cycles of biogas recirculation were varied, including the settling time, flow rate, and time of recirculation (Table 1). During Periods VIII and IX, the bioreactor broth was recirculated to reduce membrane fouling at an upflow rate of 300 mL min-1 (7.6 m h-1) or 1600 mL min-1 (40.5 m h-1) using a gear pump (M G 200-400, Fluid-o-Tech, Italy) and a variable frequency drive (JNEV-201-H1FN 4S, Teco-Westinghouse, USA). To compare the extraction efficiency between the internal and external hollow fiber membrane, the two types of pertraction were conducted in parallel during Period VII to IX (FIG. 1B, Table 1). Each period was operated for at least 20×HRT, and the average HRT and organic loading rate (OLR) were reported (FIG. 1C).

During the abiotic internal hollow fiber membrane experiments, a carboxylate synthetic solution was continuously fed to the abiotic internal hollow fiber membrane reactor. The mass transfer coefficient, and the effects of the solvent-alkaline solution interfacial area on mass transfer rate were investigated. Two interfacial areas of 62.4 cm2 and 181.8 cm2 were conducted during Stage A and Stage B, respectively. An H-type glass container and a cell culture flask were used for the abiotic pertraction experiment with interfacial areas of 62.4 cm2 and 181.8 cm2, respectively. For the biotic pertraction experiment, only the H-type glass container was used because increasing the interfacial area did not affect the mass transfer rate.

### Microbial Community Analysis

Biomass samples for Illumina 16S rRNA gene sequencing analysis were collected from the bioreactor mixed broth during Periods I to IX (Days 25, 68, 110, 137, 211, 247, 277, 325, and 380) with one sample per period. Biomass samples were collected from a sampling port that was located one-third from top of the bioreactor. The bioreactor mixed broth was collected in 2 mL centrifuge tubes and centrifuged at 10,000×g for 10 min to obtain a pellet. The obtained biomass pellets were stored at −80° C. until further analysis.

Genomic DNA extraction, DNA amplification and sequencing were performed according to the protocol in a previous study (Alqahtani, et al., Adv. Funct. Mater. 2021, 28, 1804860). Operational taxonomic unit (OTU) abundance was estimated at 97 identities using the usearch (v. 7.0.1090-usearch_global) (Bian, et al., J. Mater. Chem. A. 2021, 6, 17201-17211). Taxonomy was assigned to representative OTUs using the RDP classifier in QIIME (Caporaso et al. 2010). The following analyses were performed in R (v. 4.0.2) using the ampvis package (v.2.6.4), receiving 377 unique OTUs. Alpha diversity was analyzed using the Shannon diversity index, Simpson index and invSimpson index. Heatmap was created to represent the top 20 OTU using the ggplot package in R.

### Liquid Sampling, Analytical Procedures, and Calculations

The bioreactor broth samples were collected every other day directly from the sampling port. The samples were filtered through a 0.22-μm pore filter prior to the analyses of carboxylic acids and ethanol. The composition of carboxylic acids and ethanol was determined with a gas chromatograph (GC) (6890A Series, Agilent Technologies Inc., USA) as described previously (U sack and Angenent, Water Res. 2015, 87, 446-457). The concentrations of methane, carbon dioxide, and hydrogen in the biogas were measured weekly using a GC (model 310C; SRI Instruments, USA) as previously described (Alqahtani, et al., Adv. Funct. M ater. 2021, 28, 1804860). Detailed information on calculations is provided in the below (Eq. S1-S4).

$$Equations$$

$$\begin{matrix}Product transfer rate ( m mol {m}^{- 2} {d}^{- I} ) : \\ \underline{m} \\ S ,\end{matrix} \qquad ( Eq . S 1 )$$

where:
m=slope of the increasing specific carboxylate in the pertraction solution against time, mmol d⁻¹
S=area of hollow fiber membrane, m²
   Volumetric Production Rate (mmol C L⁻¹ d⁻¹):

$$\begin{matrix}[ ] & \underline{M} \\ HRT & V\end{matrix} \qquad ( Eq . S 2 )$$

where:
C_{e,n}=concentration of carboxylic acid in the effluent on day n, mM
HRT=hydraulic retention time on day n, d
mᵢ=slope of the increasing specific carboxylate in the pertraction solution using internal hollow fiber against time, mmol d⁻¹
mₑ=slope of the increasing specific carboxylate in the pertraction solution using external hollow fiber against time, mmol d⁻¹
M=conversion factor from mmol to mmol C; for example, acetic acid was 2 Conversion efficiency into methane (%, mM C/mM C):

$$\begin{matrix}\underline{{P}_{m}} \\ {L}_{a} + {L}_{e}\end{matrix} \qquad ( Eq . S 3 )$$

where:
Pₘ=methane production rate, mM C d⁻¹
Lₐ=acetate loading rate, mM C d⁻¹
Lₑ=ethanol loading rate, mM C d⁻¹
   Carboxylates Extraction Rates by Hollow Fiber Membrane (mmol m⁻² d⁻¹):

$$\begin{matrix}\underline{{C}_{e}} \\ M\end{matrix} \qquad ( Eq . S 4 )$$

where:
Cₑ=specific carboxylic acid extraction rate in the extraction solution, mmol d⁻¹
M=area of hollow fiber membrane, m⁻²

### Results and Discussion

Operation of Internal Hollow Fiber Model with Abiotic Synthetic Broth

The use of external hollow fiber membrane model for pertraction has been previously applied where a hydrostatic pressure of 0.5-3 psi has been used successfully in the broth side of the membrane by adjusting the valve to prevent organic solvent transferring into the fermentation broth (Kucek, et al., Water Res. 2016a, 93, 163-171; Kucek, et al., Energy Environ. Sci. 2016b, 9, 3482-3494; Kucek, et al., Front. Microbiol. 2016c, 7, 1892; Xu, et al., Chem. Commun. 2015, 51, 6847-6850; Xu, et al., Joule 2018, 2, 280-295). However, it is difficult to apply a hydrostatic pressure in the atmospheric bioreactor in this case, where the hollow fiber membrane model is submerged in the bioreactor.

To circumvent this problem, the hollow fiber membrane model was placed at the middle-to-bottom of the bioreactor (hydrostatic pressure: 0.7 psi to 1.3 psi, FIGS. 1A-1B). Steady operation was successfully achieved using abiotic synthetic broth.

Several factors affect the steady-state operation of pertraction system and the extraction rate of MCCAs, including forward and backward contactor area, the flow rate of organic solvent and alkaline solution, type of organic solvent, etc (Kucek, et al., Energy Environ. Sci. 2016b, 9, 3482-3494; Saboe, et al., Green. Chem. 2018, 20, 1791-1804). MCCA extraction by pertraction included two steps: 1) MCCA s transferring from broth to organic solvent (forward); and 2) MCCA s transferring from organic solvent to extraction solution (backward). In the backward MCCA extraction in a pertraction system, an alkaline extraction solution is used to supply a gradient as a driving force for extraction (Xu, et al., Environ. Sci. Technol. 2021, 55, 634-644). In the current study, two phases of alkaline extraction solution and organic solvent were brought in contact directly, without any membrane separator for backward extraction (FIG. 1A).

To determine whether the step of backward MCCA extraction limits the mass transfer in the pertraction system, two contactor area of 62.4 cm2 and 181.8 cm2 were applied in Stage A and B, respectively. In Stage A, the stable mass transfer of acetate, n-butyrate, n-caproate, and n-caprylate were obtained at an extraction rate of 2.3, 5.2, 13.7 and 6.3 mmol m-2 d-1, respectively (FIG. 1D). Increasing the contactor area to 181.8 cm2 in Stage B did not affect the carboxylate extraction rates (FIG. 1D), indicating that the contactor area of 62.4 cm2 for alkaline extraction solution and organic solvent was large enough for this pertraction system. Indeed, in a previous study it has been reported that the process of backward extraction was not the limiting step when using the same contactor area of forward and backward extraction. (Kucek, et al., Energy Environ. Sci. 2016b, 9, 3482-3494).

Utilizing a more apolar solvent can selectively extract longer carbon chain carboxylic acids and avoids the removal of SCCAs, which are used as a carbon source for chain elongation. In this study, a mixture of mineral oil (apolar) and 3% TOPO (polar) was used as organic solvent, which has been previously applied to extract MCCAs from fermentation reactor (Carvajal-Arroyo, et al., Chem. Eng. J. 2020, 416, 127886; Ge, et al., Environ. Sci. Technol. 2015, 49, 8012-8021; Kucek, et al., Energy Environ. Sci. 2016b, 9, 3482-3494; Urban, et al., Energ. Environ. Sci. 2017, 10, 2231-2244; Xu, et al., Chem. Commun. 2015, 51, 6847-6850; Xu, et al., Environ. Sci. Technol. 2021, 55, 634-644; Xu, et al., Joule 2018, 2, 280-295). The mineral oil has low toxicity and a food-grade of it can be used in the food industry (Saboe, et al., Green. Chem. 2018, 20, 1791-1804). It was observed that mineral oil mixed in the fermentation bioreactor for a short period did not affect the conversion processes of substrate to MCCAs. The high viscous mineral oil can lower the risk of organic solvent transferring into the bioreactor. Although higher partition coefficients of the solvents for MCCAs such as propiophenone and 2-undecanone, were previously observed (Saboe, et al., Green. Chem. 2018, 20, 1791-1804), it probably has a negative impact on conversion of substrate to MCCAs in the fermentation bioreactor once these solvents dissolved in the bioreactor. The addition of TOPO as an extractant can achieve a high equilibrium constant and increases the solvent affinity for carboxylic acid due to the polarity of its P—O bond (Carvajal-Arroyo, et al., Chem. Eng. J. 2020, 416, 127886; Saboe, et al., Green. Chem. 2018, 20, 1791-1804).

The effect of anti-membrane fouling strategies on MCCA production rate and extraction rate by internal hollow fiber membrane

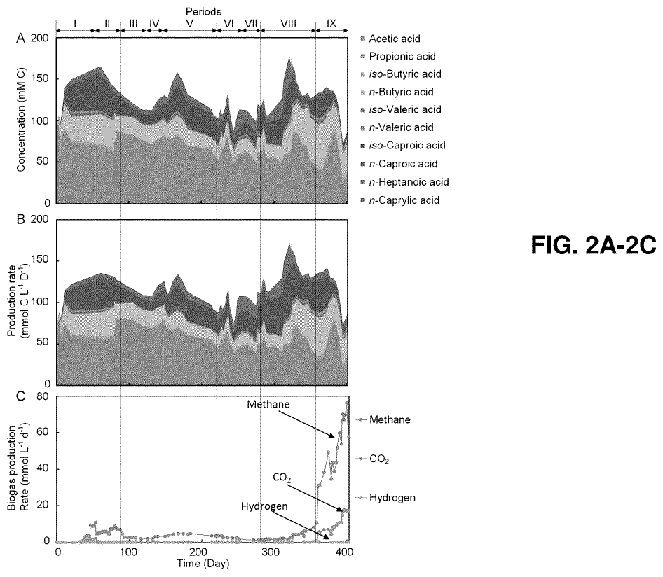

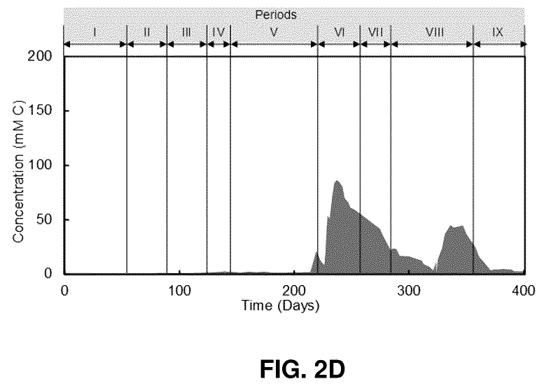

Two antifouling strategies were evaluated in this work, i.e., periodic biogas recirculation (Period II to VII) and broth recirculation (Period VIII to IX) (Table 1). During Period I, with no introduction of antifouling strategy, an extraction rate of 16.7±8.7 for n-caproate and 9.7±0.9 mmol m-2 d-1 for n-caprylate by internal pertraction was achieved. Introducing periodic biogas recirculation in Period II, resulted in an unexpected decrease in MCCA extraction rates to 11.1±1.4 and 6.3±3.5 mmol m-2 d-1 for n-caproate and n-caprylate, respectively. Several factors might have been responsible for this decrease in MCCA extraction rate as explained below. The volatile solids (VS) decreased from 5.2±0.2 to 3.9±0.01 g L-1 (FIG. 1E) due to biomass washout when biogas recirculation was applied, and this in turn might have resulted in the decrease of the concentration of n-caproate (from 31.4±13.0 to 18.2±8.9 mM C) and n-caprylate (from 6.2±2.1 to 5.2±0.4 mM C) (FIG. 2A). Decrease in MCCA extraction rate has been previously observed when the concentration of undissociated MCCAs decreased in the fermentation broth (Carvajal-Arroyo, et al., Chem. Eng. J. 2020, 416, 127886; Kucek, et al., Water Res. 2016a, 93, 161-171). The VS decrease also resulted in the decrease in MCCA production from 35.9±13.4 mmol C L⁻¹ d⁻¹ during Period I to 23.4±9.1 mmol C L⁻¹ d⁻¹ during Period II (Table 2).

TABLE 2
Bioreactor production rate and conversion efficiency during the
Periods I to IX with internal and external hollow fiber membrane.

| | Period | Period | Period | Period | Period | Period | Period | Period | Period |
|---|---|---|---|---|---|---|---|---|---|
| | I | II | III | IV | V | VI | VII | VIII | IX |
| Volumetric | 81.3 ± | 92.6 ± | 95.2 ± | 95.2 ± | 84.7 ± | 165.3 ± | 157.5 ± | 166.7 ± | 178.6 ± |
| EthOH¹ | 7.3 | 1.7 | 1.8 | 0.9 | 4.3 | 8.2 | 6.2 | 15.3 | 12.7 |
| loading rate | | | | | | | | | |
| (mmol C L⁻¹ | | | | | | | | | |
| d⁻¹) | | | | | | | | | |
| Volumetric | 40.7 ± | 46.3 ± | 47.6 ± | 42.4 ± | 41.3 ± | 39.4 ± | 41.7 ± | 41.7 ± | 44.6 ± |
| Ac¹ loading | 3.6 | 0.9 | 0.5 | 2.2 | 2.0 | 1.6 | 3.8 | 3.8 | 3.2 |
| rate | | | | | | | | | |
| (mmol C L⁻¹ | | | | | | | | | |
| d⁻¹) | | | | | | | | | |
| EthOH in | 0.7 ± | 0.7 ± | 1.7 ± | 2.7 ± | 13.9 ± | 62.1 ± | 34.1 ± | 23.3 ± | 3.5 ± |
| effluent | 0.2 | 0.4 | 0.5 | 0.3 | 3.3 | 7.4 | 10.1 | 13.4 | 0.7 |
| (mmol C L⁻¹ | | | | | | | | | |
| d⁻¹) | | | | | | | | | |
| CH₄ | 6.9 ± | 7.8 ± | 2.1 ± | 2.7 ± | 3.9 ± | 2.4 ± | 1.5 ± | 36.1 ± | 68.1 ± |
| production | 3.1 | 0.8 | 0.2 | 0.7 | 0.6 | 0.05 | 0.5 | 8.0 | 6.8 |
| rate (mmol C | | | | | | | | | |
| L⁻¹ d⁻¹) | | | | | | | | | |
| EthOH + Ac- | 5.6 | 5.6 | 1.4 | 1.8 | 3.1 | 1.1 | 0.7 | 17.3 | 30.5 |
| into-CH4 | | | | | | | | | |
| efficiency | | | | | | | | | |
| (% mmol C) | | | | | | | | | |
| CO₂ | 1.0 ± 0.4 | 0.05 ± | 0.01 ± | 0.01 ± | 0.05 ± | 0.03 ± | 0.01 ± | 5.9 ± | 17.2 ± |
| production | | 0.006 | 0.004 | 0.001 | 0.009 | 0.006 | 0.001 | 1.0 | 0.3 |
| rate (mmol C | | | | | | | | | |
| L⁻¹ d⁻¹) | | | | | | | | | |
| CA¹ | 127.5 ± | 113.3 ± | 116.3 ± | 118.8 ± | 89.4 ± | 84.5 ± | 107.2 ± | 138.8 ± | 90.6 ± 21.6 |
| production | 24.6 | 26.7 | 14.9 | 11.3 | 10.7 | 12.8 | 13.4 | 22.1 | |
| rate (mmol C | | | | | | | | | |
| L⁻¹ d⁻¹) | | | | | | | | | |
| MCCA¹ | 35.9 ± | 23.4 ± | 21.0 ± | 25.7 ± | 28.0 ± | 27.5 ± | 46.5 ± | 52.7 ± | 20.4 ± |
| Volumetric | 13.4 | 9.1 | 7.0 | 4.8 | ±7.1 | 6.2 | 6.8 | 6.3 | 9.3 |
| production | | | | | | | | | |
| rate (mmol C | | | | | | | | | |
| L⁻¹ d⁻¹) | | | | | | | | | |

¹EthOH: ethanol; AC: acetate; CA: carboxylic acid; MCCA: medium chain carboxylic acid

Periodic biogas sparging (Table 1) was continued during Period III to Period V and the highest MCCA extraction rate of 39.5 mmol m-2 d-1 was obtained during Period IV. During Period IV, the operation of biogas recirculation every 6 hr for 30 min at a flow rate of 80 min min-1 and ethanol: acetate of 50:25 (mol: mol) was considered the optimum condition for MCCA extraction in this system. Biogas recirculation was applied in submerged membrane system not only to scour the outer membrane surface and induce a shear force at the membrane surface to remove the accumulated foulants (Fulton, et al., Desalination 2011, 281, 128-141; Vermaas, et al., Environ. Sci. Technol. 2014, 48, 3065-3073), but also to induce a turbulent flow which can increase mass transfer rate (Laptev, et al., J. Eng. Phys. Thermophy. 2015, 88, 207-213). Mathematical modelling analysis should be used in future studies to describe and understand the effect of the performing conditions on the process of mass transfer.

To increase the MCCA production rate, the influent concentration of ethanol was doubled to 100 mM during Period VI (Table 1). Enough ethanol (75.2±8.9 mM C) was present in the broth as an electron donor and carbon source to sustain a promising chain elongation rate (FIG. 2D). The average MCCA extraction rate of 97.4 mmol m-2 d-1 (62.0±6.0 mmol C6 m-2 d-1 and 35.4±6.3 mmol C8 m-2 d-1) obtained here were higher than the rates reported in previous chain elongation studies using external membrane pertraction (Table 3).

Carvajal-Arroyo et al (Carvajal-Arroyo, et al., Chem. Eng. J. 2020, 416, 127886) reported a MCCA mass flux of 11.1 g m-2 d-1 (the aggregated reported number of MCCAs does not allow comparison with a molar unit), while an average maximum MCCA mass flux of 12.2 g m-2 d-1 was achieved in the present study which was slightly higher than that obtained in Carvajal-Arroyo et al. A regular offline cleaning (once every 3-5 weeks) for the external membrane model by flushing with water to remove the accumulated foulants was performed in previous studies (Xu, et al., Chem. Commun. 2015, 51, 6847-6850; Xu, et al., Environ. Sci. Technol. 2021, 55, 634-644), while a continuous high extraction rate was achieved in this work at least for 248 days by regularly recirculating without any offline washing or application of anti-fouling chemical agents. The broth biomass concentration in this work (FIG. 1E) was relatively lower than most previous studies (Ge, et al., Environ. Sci. Technol. 2015, 49, 8012-8021; Xu, et al., Joule 2018, 2, 280-295), and it has been reported that increase in biomass could result in an increase in conversion rate and concentration of products (Xu, et al., Joule 2018, 2, 280-295), thus further leading to high extraction rate (Carvajal-Arroyo, et al., Chem. Eng. J. 2020, 416, 127886; Kucek, et al., Water Res. 2016a, 93, 163-171). Therefore, increasing the biomass concentration (e.g., adding carriers) would result in further increase in MCCA extraction rate using internal pertraction, albeit probably negatively affecting membrane fouling.

TABLE 3
Performance parameters and MCCA extraction rates of external
membrane model pertraction system from previously published studies
which were similar to the one used in the present study.

| | | Ratio of | | n- | n- | |
|---|---|---|---|---|---|---|
| | | membrane | Broth | Caproate | Caprylate | MCCAs |
| | | area to | recycle | extraction | extraction | extraction |
| | Type | reactor | flow | rate | rate | rate |
| Refer- | of the | volume | rate (m | (mmol | (mmol m⁻² | (mmol |
| ence | broth | (m² L⁻¹) | hr⁻¹) | m⁻² d⁻¹) | d⁻¹) | m⁻² d⁻¹) |
| 6 | Filtered | 2.5 | 1.1 | 5.3 | 0.7 | 6.8 |
| | bioreactor | | | | | |
| | broth | | | | | |
| 5 | Filtered | 0.4 | 1.0 | 11.4 | 11.1 | 22.5 |
| | bioreactor | | | | | |
| | broth | | | | | |
| 4 | Abiotic | 11.6 | 9.5 | 6.6 | — | — |
| | synthetic | | | | | |
| | broth | | | | | |
| 4 | Filtered | 2.0 | 3.4 | 3.0 | 27.2 | 30.2 |
| | bioreactor | | | | | |
| | broth | | | | | |
| 1 | Filtered | 0.14 | 1.9 | 57.8 | — | 11.1 |
| | bioreactor | | | | | g m⁻² |
| | broth | | | | | d⁻¹ |
| 3 | Filtered | 2.5 | 1.6 | 10.5 | — | 10.5 |
| | bioreactor | | | | | |
| | broth | | | | | |
| 2 | Filtered | 1.6 | — | 17.7 | — | 17.7 |
| | bioreactor | | | | | |
| | broth | | | | | |

During Period VIII, the broth was recirculated at a flow rate of 300 ml min-1 (upflow velocity of 7.6 m h-1) to reduce hollow fiber membrane fouling with an internal and external hollow fiber membrane pertraction operated in parallel (Table 1). A higher MCCA extraction rate (31.2 mmol m-2 d-1) was observed in Period VIII (broth recirculation with two types of pertraction operated in parallel) compared to 24.3 mmol m-2 d-1 during Period VII (biogas recirculation with two types of pertraction operated in parallel) (Table 4).

TABLE 4
Carboxylates extraction rates with two pertraction
strategies during the nine periods.

| | | | | | |
|---|---|---|---|---|---|
| | Acetate | n-Butyrate | n-Caproate | n-Caprylate | MCCAs |
| | extraction | extraction | extraction | extraction | extraction |
| | rate (mmol | rate (mmol | rate (mmol | rate (mmol | rate (mmol |
| Periods | m⁻² d⁻¹) | m⁻² d⁻¹) | m⁻² d⁻¹) | m⁻² d⁻¹) | m⁻² d⁻¹) |

Internal pertraction

| | | | | | |
|---|---|---|---|---|---|
| I | 9.7 ± 1.2 | 11.2 ± 5.2 | 16.7 ± 8.7 | 9.7 ± 0.9 | 26.4 ± 9.6 |
| II | 2.6 ± 1.1 | 1.3 ± 0.4 | 11.1 ± 1.4 | 6.3 ± 3.5 | 17.4 ± 4.9 |
| III | 7.3 ± 3.1 | 6.0 ± 1.4 | 20.3 ± 7.7 | 6.7 ± 0.3 | 27.0 ± 8.0 |
| IV | 6.1 ± 0.13 | 8.8 ± 1.1 | 20.6 ± 7.4 | 18.9 ± 4.2 | 39.5 ± 11.6 |
| V | 1.6 ± 0.5 | 1.6 ± 0.6 | 14.8 ± 2.2 | 6.1 ± 1.4 | 20.9 ± 3.6 |
| VI | 9.6 ± 5.9 | 14.3 ± 7.6 | 62.0 ± 6.0 | 35.4 ± 6.3 | 97.4 ± 12.3 |
| VII | 5.9 ± 2.0 | 5.9 ± 1.8 | 14.0 ± 8.0 | 10.3 ± 5.5 | 24.3 ± 13.5 |
| VIII | 7.3 ± 1.4 | 7.3 ± 0.8 | 18.7 ± 6.2 | 12.5 ± 3.7 | 31.2 ± 9.9 |
| IX | 3.0 ± 1.5 | 3.8 ± 3.0 | 5.0 ± 1.9 | 10.0 ± 1.7 | 15.0 ± 3.6 |

External pertraction

| | | | | | |
|---|---|---|---|---|---|
| VII | — | 0.56 ± 0.14 | 5.7 ± 0.9 | 2.0 ± 0.2 | 7.7 ± 1.1 |
| VIII | 0.2 ± 0.02 | 1.1 ± 0.1 | 9.4 ± 1.1 | 1.0 ± 0.1 | 10.4 ± 1.2 |
| IX | 0.14 ± 0.03 | 0.33 ± 0.08 | 2.0 ± 0.6 | 1.2 ± 0.4 | 3.2 ± 1.0 |

The results indicated that the extraction rate during operation with broth recirculation was higher than operation with biogas recirculation when internal and external membrane pertraction were operated in parallel. Even though similar fouling strategy (biogas recirculation rate and frequency) was applied in Period VI and VII and similar ethanol: acetate ratio (mol: mol), the extraction rate was significantly higher in Period VI (97.4. mmol m-2 d-1) than Period VII (24.3 mmol m-2 d-1), possibly because only internal hollow fiber membrane pertraction (1.5% membrane area of external membrane area) was applied in Period VI compared to internal and external in Period VII. Increasing the broth recirculation rate to 1600 mL min-1 (40.5 m h-1) in Period IX resulted in an obvious decrease in MCCA extraction rate to 15.0 mmol m-2 d-1. Under a higher broth up-flow velocity, the concentrations of n-caproate and n-caprylate in the broth decreased to 7.6 mM C and 3.9 mM C, respectively (FIG. 2A). The decrease in extraction rate could be due to the low MCCA concentration in the broth, where more substrates were converted to methane than MCCA (FIG. 2C).

### Comparison of Internal and External Pertraction on MCCA Extraction Rate and Production Rate

To evaluate the extraction efficiency of internal pertraction, an external pertraction was set up and operated in parallel with internal pertraction to extract MCCAs from the fermentation reactor during Period VII to IX. During these periods, the extraction rate of n-caproate and n-caprylate by internal pertraction was 2.0- to 2.5-fold and 5.2- to 12.5-fold higher than by external pertraction, respectively (Table 4). The results indicated that the MCCA extraction efficiency by internal pertraction was much higher than by external pertraction with the same chain elongation bioreactor (FIG. 1B). It has been reported that MCCA mass transfer limitations were at the interface of the fermentation broth and the hydrophobic membrane contactor in the external pertraction system, which was similar to the one used in the present work, and increasing the recycle flow rate of broth increased MCCA mass transfer (Kucek, et al., Energy Environ. Sci. 2016b, 9, 3482-3494). In the current study, a broth recycle flow rate of 3 L hr-1 (1.6 m hr-1) was applied in the external pertraction system which was similar to broth recycle flow rates of 1.5-5.8 L hr-1 (0.9-3.4 m hr-1) used previous studies (Carvajal-Arroyo, et al., Chem. Eng. J. 2020, 416, 127886; Kucek, et al., Energy Environ. Sci. 2016b, 9, 3482-3494; Xu, et al., Environ. Sci. Technol. 2021, 55, 634-644; Xu, et al., Joule 2018, 2, 280-295). In the internal pertraction system, both biogas recirculation (Period VII) and broth recirculation (flow rate of 300 ml min-1 in Period VIII and 1600 mL min-1 in Period IX) were used to minimize fouling of the membrane and increase mass transfer. Thus, biogas recirculation or higher broth recirculation flow rate could result in higher MCCA mass transfer from the fermentation broth to the hydrophobic solvent. The lower footprint and energy consumption are additional advantages of using internal pertraction system compared to external pertraction system, which requires heating of the recycle broth from external membrane The MCCA production rate was increased from 27.5 mmol C L⁻¹ d⁻¹ during Period VI (biogas recirculation only) to 46.5 mmol C L⁻¹ d⁻¹ during Period VII (broth recirculation only), and the highest production rate of 52.7 mmol C L⁻¹ d⁻¹ was obtained during Period VIII (Table 2).

These results indicate that continuous pertraction can lead to an increase in MCCA production rate due to reducing the MCCA cell toxicity and end-product feedback inhibition in the fermentation bioreactor.

The ratio of pertraction membrane area-to-reactor volume for internal pertraction was only 0.004 m2 L-1, which was much lower than the ratio (0.35 to 2.5 m2 L-1) for external pertraction reported in previous studies (Kucek, et al., Water Res. 2016b, 93, 163-171; Xu, et al., Environ. Sci. Technol. 2021, 55, 634-644; Xu, et al., Joule 2018, 2, 280-295). In the current study, the MCCA extraction efficiency using internal pertraction was only 0.5-3.8% during all periods. Therefore, the ratio of pertraction membrane area-to-reactor volume was increased to 0.33 m2 L-1 by operating an external pertraction model in parallel with the internal pertraction system during Period VII. The MCCA production rate was increased from 27.5 mmol C L-1 d-1 during Period VI (biogas recirculation only, no external pertraction) to 46.5 mmol C L-1 d-1 during Period VII (biogas recirculation only, with external pertraction), and the highest production rate of 52.7 mmol C L-1 d-1 was obtained during Period VIII (broth recirculation only) (FIG. 2B; Table 2). These results indicate that continuous pertraction can lead to an increase in MCCA production rate due to reducing the MCCA cell toxicity and end-product feedback inhibition in the fermentation bioreactor.

### The Effect of Anti-Membrane Fouling Strategies on Biomass Concentration and Microbial Community Composition

In the current study, the VS concentration in the fermentation bioreactor decreased from 5.2±0.2 to 3.9±0.01 g L-1 (FIG. 1E) when biogas recirculation was applied during Period II. The VS remained stable at 3.6-4.0 g L-1 during Period II to VII with different biogas recirculation frequency, duration, and flow rate. The VS concentration significantly decreased from 3.6±1.1 g L-1 to 1.5±0.2 g L-1 when broth recirculation rate of 300 ml min-1 (Period VIII) was applied. High biomass concentration in the fermentation bioreactor is commonly considered to achieve high production rates (Carvajal-Arroyo, et al., Green. Chem. 2019, 21, 1330-1339). High concentration of biomass in the chain elongation reactor can be achieved by i) using packing material or settlers (Grootscholten, et al., Bioresour. Technol. 2013, 136, 735-738; Kucek, et al., Energy Environ. Sci. 2016b, 9, 3482-3494; Liu, et al., Water Res. 2017, 119, 150-159); ii) forming a chain elongation granular sludge (Carvajal-Arroyo, et al., Green. Chem. 2019, 21, 1330-1339; Roghair, et al., Process Biochem. 2016, 51, 1594-1598); and iii) using a membrane to prevent biomass washout (Kim, et al., Bioresour. Technol. 2018, 270, 498-503). Therefore, improving reactor design to enhance biomass retention would result in a higher production rates, however, higher biomass concentration might enhance membrane fouling and future studies should evaluate the maximum biomass concentration required to achieve good production rate and MCCA extraction by internal hollow fiber membrane without elevating membrane fouling.

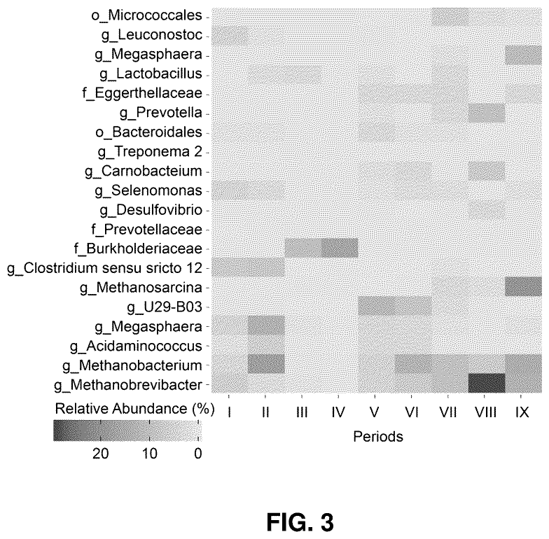

Methanogens exist in nearly every conceivable anaerobic environment and organisms can convert organic substrates effectively into methane because it has the lowest free energy content per electron (Angenent, et al., Environ. Sci. Technol. 2016, 50, 2796-2810; Zinder, “Physiological Ecology of Methanogens,” in Methanogenesis: Ecology, Physiology, Biochemistry & Genetics. Editor J. G. Ferry (Boston, MA: Springer), 1993, 128-206). To establish an MCCA production process in open culture fermentations, one successful option for inhibition of methanogenic activity was maintaining an acidic pH of approximately 5.5 in the fermentation broth (Kucek, et al., Front. Microbiol. 2016c, 7, 1892; Xu, et al., Chem. Commun. 2015, 51, 6847-6850; Xu, et al., Joule 2018, 2, 280-295). In the current study, the pH was maintained at 5.5 and conversion efficiency into methane was low (0.7-5.6% of ethanol and acetate conversion into methane, mol C/mol C; FIG. 2C; Table 2; Eq. S3) during Period I to Period VII (biogas recirculation) despite the fact that members of hydrogenotrophic methanogens belonging to the genus Methanobacterium and Methanobrevibacter were predominant (accounting for 9.0-19.9% of the total reads) (FIG. 3). During Period VIII (broth recirculation at an up-flow velocity of 7.6 m hr-1), members of the genus Methanobrevibacter (relative abundance of 28.9%), Prevotella (relative abundance of 9.0%) and Methanobacterium (relative abundance of 6.7%) were the predominant OTUs detected in the bioreactor (FIG. 3). The conversion efficiency to methane increased to 17.3% (mol C/mol C, Table 2) in Period VIII. When the broth up-flow velocity was increased to 40.5 m hr-1 (Period IX), methane production rate significantly increased in the biogas (FIG. 2C) and conversion efficiency to methane increased to 30.5% (mol C/mol C, Table 2). It should be noted that the highest relative abundance (46.7%) of methanogens (Methanobrevibacter, Methanobacterium, and Methanosarcina) was detected in Period IX. These results indicate that at high upflow velocity the microbial community shifted towards methanogens. Different bacterial cells experience a different response to physical force (Dufrêne and Persat 2020). Therefore, it was hypothesized that chain elongation microbes were more sensitive to mechanical force generated by fluid flow and pressure as well as surface contact compared to methanogens, and this resulted in the shift in the bioprocess towards methanogenesis.

### Conclusions

A submerged hollow fiber membrane (internal) in the fermentation bioreactor was able to achieve high MCCA extraction rate for a long period by biogas recirculation without any offline washing or anti-fouling chemical agent application to remove foulants. However, higher broth up-flow velocity led to low concentration of MCCAs in the fermentation broth because of shift in conversion towards methane production. The results obtained here showed that the extraction rate of MCCAs by internal pertraction was much higher than by external pertraction (traditional pertraction) in the same bioreactor. The results in this work showed that the concentration of biomass in this system was relatively low. The use of biocarriers may also help in reducing membrane biofouling.

### Example 2

Fruits, vegetables, and dairy products are typically the primary sources of household food waste. Currently, anaerobic digestion is the most used bioprocess for the treatment of food waste with concomitant generation of biogas. However, to achieve a circular carbon economy, the organics in food

waste should be converted to new chemicals with higher value than energy. So far, the most common dairy waste treated via lactate-driven chain elongation is acid whey and cheese whey. Different pH, hydraulic retention times, MCCA extraction methods, and single-stage or two-stage processes have been explored with lactate-driven microbial chain elongation.

Considering the significantly higher concentrations of fat and proteins (˜3.5%, of which 80% are casein proteins and 20% whey proteins [6]) in expired milk/yogurt/beverage waste and the associated higher treatment challenges compared to acid/cheese whey wastes, it is thus essential to

develop a two-stage chain elongation platform, allowing the investigation of operation conditions separately in each stage to achieve satisfactory MCCA production.

The present studies demonstrate the feasibility of medium-chain carboxylic acid (MCCA) production from expired dairy and beverage waste via a chain elongation platform mediated by lactate.

### Materials and Methods

### Fermentation Reactor Construction and Operation

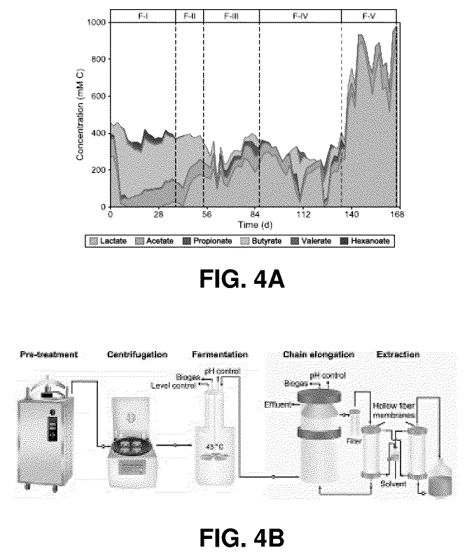

A fermentation reactor (5 L working volume) with a height of 28 cm and internal diameter of 19.5 cm was established and inoculated with 200 mL anaerobic sludge from a continuous stirred tank reactor (CSTR). Expired milk, yogurt, and fruit (mainly orange) juice (total level of chemical oxygen demand (COD)>200 g L-1) received from a local food company were mixed at a ratio of 5:3.2:1.8 based on the actual percentage of each expired food in the total waste and then fed without any pretreatment to the fermentation reactor that was operated in batch mode for initial acclimatization. After one month of batch operation, the fermentation reactor was operated in continuous mode without biomass recirculation or settling and fed with the waste mixture that was pretreated at 100° C. for 20 min to remove the excessive protein content through centrifugation at 4000×g for 10 min (FIG. 4b). The centrifuged medium (CM, total COD level 50-80 g L⁻¹) was then stored at 4° C. and used as a feedstock for the fermentation reactor.

Continuous operation of the first-stage fermentation reactor was divided into five periods (F-I, F-II, F-III, F-IV, and F-V) to optimize operation conditions (i.e., temperature and organic loading rate) for enhancing lactate concentration and production rate. The optimization details, including increasing the operation temperature (from 30 to 35 and then 43° C., F-I to F-III) and the organic loading rate by adjusting the hydraulic retention time (HRT, F-IV) and CM dilution ratio (F-V), could be found in Table 5. The pH in the fermentation reactor was maintained at 5.2 by periodically adding sodium hydroxide solution (1 M), and the agitation was fixed at 100 rpm by a control panel (Huisen Bio, China). Chain elongation reactor construction and operation

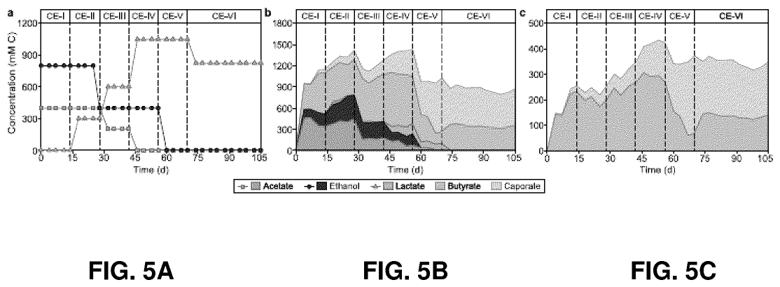

The chain elongation reactor was scaled up to a working volume of 10 L from an up-flow reactor (2.25 L), with an internal diameter of 11 cm and a height of 126 cm. The ethanol/acetate mixture was used as the initial feed substrates and electron donors for the enrichment of chain elongation microbiome and MCCA production. To examine the impacts of different feed substrates on MCCA production, microbial community evolution, and the feasibility of a two-stage system for MCCA from expired dairy/beverage waste, the operation of the chain elongation reactor was divided into six periods (CE-I to CE-VI, Table 5), by gradually switching the feed substrate from ethanol/acetate to lactate only, and then to the effluent from the fermentation reactor above. The details of feed substrates during Periods CE-I to CE-VI can be found in FIG. 5A, Table 5. The chain elongation reactor was initially inoculated in Period CE-I with a mixture of activated sludge from the municipal Wastewater

Treatment Plant at King Abdullah University of Science and Technology, anaerobic sludge from a lab-scale anaerobic membrane bioreactor, and sediments from a lab-scale fermentation reactor. An additional source of inoculum using local sheep rumen was added between Periods CE-III and CE-IV to examine the impact of inocula on the MCCA production and microbial community evolution in the second-stage chain elongation reactor. Plastic biocarriers were added to the chain elongation reactor to improve biomass retention. The HRT for the chain elongation reactor was set at 2.5 days, and 1-3 g L⁻¹ yeast extract was added during the whole experiment except in Period CE-VI based on previous studies. The temperature was maintained at 35° C. using a recirculating water bath (MP-5H, Hinotek, China). The broth pH was maintained at 5.5-6.0 by an automatic pH controller (400 pH/ORP, Cole-Parmar, the United States) and a dosing pump to add sodium hydroxide solution (1 M) at the top of the reactor. The biogas was collected and recorded by a flow gas meter (TG05, Ritter, Germany). An external extraction unit made of hollow-fiber membranes was constructed for the continuous forward and reverse extraction of MCCAs from the chain elongation bioreactor.

### Microbial Community Analysis

Biomass samples were collected from the fermentation reactor before switching to continuous-flow operation and at the end of each period of continuous-flow operation. Similarly, biomass samples of the original inoculum, rumen inoculum, and at the end of each period (except CE-II and CE-III) were collected for the chain elongation reactor. The variable region 4-8 (abeV 48-A) of the archaea/bacteria/eukarya 16S/18S rRNA gene was sequenced using a custom protocol. The detailed procedure for DNA extraction, sequencing library preparation, sequencing, and processing of sequence reads. Co-occurrence network analysis was conducted for the microbial communities collected from the fermentation and chain elongation reactor during Periods F-I to F-VI and CE-I to CE-VI, respectively. Multiple testing correction was conducted via false discovery rate (FDR) estimation using “igraph” and “psych” packages in R Studio. The correlations between operational taxonomic units (OTUs) were considered significant when Spearman's correlation coefficient r>0.6 and p<0.01. The fast-greedy modularity optimization was applied to calculate the modular structure of the phylogenetic molecular ecological networks of microbial communities in the fermentation and chain elongation reactors.

TABLE 5
Experimental approach and operating conditions for the
fermentation and chain elongation reactors

| Operation parameters for the fermentation reactor | | | | | Organic |
|---|---|---|---|---|---|
| | | Temper- | | Medium | loading |
| | | ature | HRT | dilution | rate (g |
| Periods | Days | (C) | (d) | (CM:TW)^{a} | COD L¹ [symbol] |
| D | 0-38 | 30 | 4 | 1:2 | 4.9 |
| F-II | 39-54 | 35 | 4 | 1:2 | 5.1 |
| F-III | 55-86 | 43 | 4 | 1:2 | 4.2 |
| F-IV | 87-134 | 43 | 2 | 1:2 | 8.4 |
| FeV | 135-166 | 43 | 2 | CM without dilution | 28.1 |
| Operation parameters for the chain elongation reactor | | | | | Organic |
| | | Temper- | | | loading |
| | | ature | HRT | Medium | rate (g |
| Periods | Days | (C) | (d) | (mM C)b | COD L1 d 1) |
| CE-I | 0-14 | 35 | 2.5 | EtOH 800, Ac 400 | 25.7 |
| CE-II | 15-28 | 35 | 2.5 | EtOH 800, Ac 400, | 29.4 |
| | | | | LA 300 | |
| CE-III | 29-42 | 35 | 2.5 | EtOH 400, Ac 200, | 25.7 |
| | | | | LA 600 | |
| CE-IV | 43-56 | 35 | 2.5 | EtOH 400, LA 1050 | 28.0 |
| CE-V | 57-70 | 35 | 2.5 | LA 1050 | 18.2 |
| CE-VI | 71-105 | 35 | 2.5 | Effluent from | 19.2 |
| | | | | fermentation | |
| | | | | reactor containing | |
| | | | | LA 810e840 | |

^{a}CM: centrifuged medium, TW: tap water.
bEtOH: ethanol; Ac: acetate; LA: lactic acid. [symbol]
indicates data missing or illegible when filed

### Liquid and Gas Sample Analysis

Liquid samples were collected every two days from the fermentation reactor and twice a week from the chain elongation reactor and filtered using 0.22 mm syringe filters (VWR). High performance liquid chromatography (HPLC, Waters) was used for the detection of volatile fatty acids (VFAs) and lactate in the filtrate. Since the exact component composition in the fermentation-influent mixture (i.e., expired milk/yogurt/juice) is not known, the average selectivity of each product in the fermentation effluent was calculated by dividing the concentration of the formed product by the total product concentration (based on mM C), while in the chain elongation reactor it was calculated by dividing the concentration of electrons in the formed products (butyrate and caproate) by the net consumed electrons from lactate. Gas chromatograph-mass spectrometry (GC-MS, 7890A, Agilent Technologies) equipped with a flame ionization detector (FID) was used for the detection of ethanol in the filtrate. Gas samples were periodically analyzed using gas chromatography (GC, model 310C, SRI Instruments).

### Reactor Construction and Operation

### Continuous Operation of Fermentation Reactor

For the continuous operation of the fermentation reactor, first, three different temperatures were tested for their impact on lactate concentration and production rate using a heating jacket: Period F-I (30° C.), Period F-II (35° C.), and Period F-III (43° C.). The CM medium stored at 4° C. was diluted with tap water (TW) at a ratio of 1:2 before being used as the influent to the fermentation reactor in Period F-I to F-III, and the hydraulic retention time (HRT) was 4 days. Next, the effect of increasing the organic loading rate on the performance of the fermentation reactor was examined. The HRT was adjusted to 2 days in Period F-IV and F-V, and to further increase the organic loading rate CM with no dilution was used as a feedstock in Period F-V.

### Feed Substrates During Periods CE-I to CE-VI in Chain Elongation Reactor

Synthetic medium containing 800 mM-C ethanol (EtOH, Sigma-Aldrich) and 400 mM-C sodium acetate (Ac, Sigma-Aldrich) was initially fed into the reactor in Period CE-I to enrich for chain elongation microbes. Given that the main product in the fermentation reactor was lactate, the influent for the chain elongation reactor was adjusted in Periods CE-II to CE-V by gradually adding DL-lactic acid (LA, Sigma-Aldrich) to 1050 mM-C and reducing the concentration of ethanol and acetate to 0 mM-C in Period CE-V. In Period CE-VI, unfiltered and unsterilized fermentation reactor effluent, containing around 810-840 mM-C lactate, was used as the feed for the chain elongation reactor, to test the feasibility of chain elongation platform for the production of MCCAs through a two-stage fermentation process using expired dairy and beverage waste as raw materials.

MCCA Extraction from Chain Elongation Reactor

One external extraction system made of hollow-fiber membranes was constructed for the continuous forward and reverse extraction of MCCAs from the chain elongation bioreactor, similar to previous reports [1]. The chain elongation broth was continuously circulated through the exterior space of the forward membrane module at a flow rate of 50 ml min⁻¹. Mineral oil solvent (VWR) with 3% tri-n-octylphosphine oxide (TOPO) (Alfa Aesar) was used as the hydrophobic forward extraction solvent at an upflow rate of 30 ml min⁻¹, while the alkaline solution buffered with 0.2 M boric acid was utilized for the reverse extraction, which was maintained at a pH of 9-11 with manual addition of 2 M sodium hydroxide solution every 4-7 days.

### DNA Extraction and Sequencing

### DNA Extraction

DNA extraction of the samples was done using a slightly modified version of the standard protocol for FastDNA Spin kit for Soil (MP Biomedicals, USA) with the following exceptions: 500 μL of sample, 480 μL Sodium Phosphate Buffer and 120 μL MT Buffer were added to a Lysing Matrix E tube. Bead beating was performed at 6 m/s for 4×40s [2]. Gel electrophoresis using Tapestation 2200 and Genomic DNA screentapes (Agilent, USA) was used to validate product size and purity of a subset of

DNA extracts. DNA concentration was measured using Qubit dsDNA HS/BR Assay kit (Thermo Fisher Scientific, USA).

### Sequencing Library Preparation

Amplicon libraries for the archaea/bacteria/eukarya 16S/18S rRNA gene variable regions 4-8 (abeV 48-A) were prepared using a custom protocol. Up to 25 ng of extracted DNA was used as template for PCR amplification of the archaea/bacteria/eukarya 16S/18S rRNA gene variable regions 4-8 (abeV 48-A). Each PCR reaction (50 μL) contained 0.5 mM dNTP mix, 0.01 units of Platinum SuperFi DNA Polymerase (Thermo Fisher Scientific, USA), and 500 nM of each forward and reverse primer in the supplied SuperFI Buffer. PCR was done with the following program: Initial denaturation at 98° C. for 3 min, 25 cycles of amplification (98° C. for 30 s, 62° C. for 20 s, 72° C. for 2 min) and a final elongation at 72° C. for 5 min. The forward and reverse primers used include custom 24 nt barcode sequences followed by the sequences targeting the archaea/bacteria/eukarya 16S/18S

rRNA gene variable regions 4-8 (abeV 48-A): [515FB]

### GTGYCAGCMGCCGCGGTAA (SEQ ID NO:1) and [1391R]

GACGGGCGGTGWGTRCA 9 SEQ ID NO: 2) [3, 4]. The resulting

amplicon libraries were purified using the standard protocol for CleanNGS SPRI beads (CleanNA, NL) with a bead to sample ratio of 3:5. DNA was eluted in 25 μL of nuclease free water (Qiagen, Germany). Sequencing libraries were prepared from the purified amplicon libraries using the SQK-L SK 110 kit (Oxford Nanopore Technologies, UK) according to manufacturer protocol with the following modifications: 500 ng total DNA was used as input, and CleanNGS SPRI beads for library clean-up steps. DNA concentration was measured using Qubit dsDNA HS Assay kit (Thermo Fisher Scientific, USA). Gel electrophoresis using Tapestation 2200 and D 1000/High sensitivity D 1000 screentapes (Agilent, USA) was used to validate product size and purity of a subset of amplicon libraries.

### DNA Sequencing

Circa 12 ng (˜20 fmol) of the resulting sequencing library was loaded onto a MinION R10.4.1 flowcell and sequenced using the MinKNOW v22.03.6 software (Oxford Nanopore Technologies, UK). Reads were base-called and demultiplexed with MinKNOW guppy v. 6.0.7 using the super accurate basecalling algorithm (configr10.4.1_450bps_sup.cfg) and custom barcodes.

### Bioinformatic Processing of Sequence Reads

Sequencing reads in the demultiplexed and basecalled fastq files were length filter for length (320-2000 bp) and quality (phred score >15) using a local implementation of filtlong v0.2.1 with the settings -min_length 700-max_length 2000-min_mean_q 97. The filtered reads were mapped to the QIIME-formatted MiDAS database, release 4.8.1 [5-8] with minimap2 v2.24-r1122 using the -ax map-ont command [9] and downstream processing using samtools v1.14 [10]. Mapping results were filtered such that query sequence length relative to alignment length deviated <5%. Noteworthy, low abundant operational taxonomic units (OTUs) making up <0.01% of the total mapped reads within each sample were disregarded as a data denoising step. Further bioinformatic processing was done via R Studio

IDE (2022.2.3.492) running R version 4.2.2 (2022-10-31) and using the R packages: ampvis2 (2.7.27) [1], tidyverse (1.3.1), seqinr (4.2.16), ShortRead (1.54.0) and iNEXT (2.0.20) [11, 12].

### Results and Discussion

### Effect of Temperature on the Performance of the Fermentation Reactor

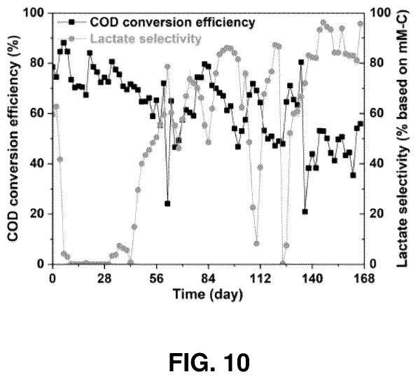

The temperature had a notable effect on the product spectrum in the fermentation reactor (FIG. 4A), switching the main component from butyrate at 30° C. (Period F-I) to lactate at 43° C. (Period F-III). The maximum concentration of butyrate (380.4±10.8 mM C) at 30° C. (Period F-I) was achieved on day 6 with the diluted medium (CM: tap water (TW) ¼ 1:2), representing a production rate of 95.1±2.7 mM C d⁻¹ at an HRT of 4 d (FIG. 4A) and a butyrate selectivity of 87.2±0.2% (based on mM C) within all lactate and VFAs in Period F-I (30° C.). However, the lactate production was severely suppressed (18.3±0.3 mM C on day 6) at 30° C. with selectivity usually below 10% in the fermentation effluent (FIG. 10), which was reported to be insufficient for the efficient MCCA production through chain elongation. Apart from lactate and butyrate, acetate (17.8±0.5 mM C) and propionate (19.7±3.4 mM C) were also measured. Considering 19.4 g COD L-1 in the influent, the maximum COD conversion efficiency to VFAs and lactate reached 88.0±6.9% on day 6 at 30° C. (Period F-I, FIG. 10), which was slightly higher than a previous report using acid whey as the feedstock.

After switching the operation temperature to 35° C. (Period F-II) and then 43° C. (Period F-III), the product spectrum was dramatically shaped, as the average butyrate concentration after stabilization dropped from 263.9±44.1 mM C at 30° C. to as low as 38.6±14.8 mM C at 43° C. (t-test, p value <0.01). On the contrary, the lactate concentration significantly increased from almost 0 mM C to an average of 187.9±49.0 mM C (t-test, p value <0.0001), with the highest concentration reaching 264.9±6.3 mM C on day 78 in Period F-III (FIG. 4A). The average lactate concentration could be maintained at 187.6-264.9 mM C after stabilization at 43° C. (Period F-III), which was reported to be sufficient to initiate the chain elongation process. 50.7±1.2% of the influent COD was converted to lactate on day 78, and the highest lactate selectivity (73.8±3.6% based on mM C) in the effluent was obtained on day 74 at 43° C. The average lactate selectivity at 43° C. (63.8±9.7%, Period F-III) was significantly higher compared to 30° C. (0.7±1.5%, Period F-I, FIG. 10) (t-test, p value <0.01), but was slightly lower than that reported in literature using acid whey for the lactate fermentation at 50° C. and close to what was achieved by Bühlmann et al. with food waste mix at 40-50° C. The improved lactate production and selectivity at 43° C. during Period F-III indicated the potential change in the fermentation pathways. Homolactic fermentation was believed to play a major role in lactate production at 43° C. during Period F-III with the enrichment of homolactic microorganisms, such as Lactobacillus and Lacticaseibacillus.

However, apart from the main product lactate, 12.0-96.2 mM C of acetate and 0-61.1 mM C of propionate were also observed in the effluent at 43° C. (Period F-III), indicating the potential involvement of heterolactic fermentation or lactate-consuming pathways involved at 43° C. The heterolactic fermentation or lactate-consuming pathways was partially demonstrated by the higher production of H₂ and CO₂ as byproducts with butyrate and acetate during Period F-I (1.3 L CO₂ d⁻¹ and 1 L H₂ d⁻¹) and F-II (0.9 L CO₂ d⁻¹ and 0.8 L H₂ d⁻¹), compared to 0.4 L CO₂ d⁻¹ and 0.2 L H₂ d⁻¹ during Period F-III in the gas phase. The maximum percentage (74.3±2.6% to 79.6±2.6%) of COD (16.7 g COD L⁻¹ in the influent) converted to VFAs and lactate occurred between day 78 and day 84 and was slightly lower than what was obtained at 30° C. (Period F-I), which as mainly due to the lower COD conversion factor of 1 g lactic acid (1.066 g COD) compared to 1 g butyric acid (1.816 g COD). Increasing the operation temperature for the fermentation reactor in this study improved the lactate production/selectivity and potentially enhanced MCCA production in the following chain elongation reactor.

### Effect of Increasing the Organic Loading Rate on the Performance of the Fermentation Reactor

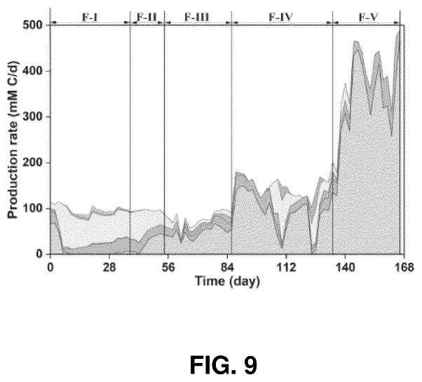

Increasing the organic loading rate to further enhance the lactate production was first demonstrated by reducing the HRT from 4 days during Period F-III to 2 days during Period F-IV (both at 43° C. and medium dilution CM: TW ¼ 1:2). The lactate concentration immediately increased from 220.5±1.6 to 274.4-296.3 mM C (days 88-92) after reducing the HRT. The highest lactate concentration of 296.3±7.6 mM C was obtained on day 90 in Period F-IV (HRT ¼ 2 d) (FIG. 4), representing a 35% increase compared to Period F-III (HRT ¼ 4 d). Similarly, the production rate of lactate increased by 124%, from 66±2 mM Cd-1 on day 78 in Period F-III (HRT ¼ 4 d) to 148±4 mM C d⁻¹ on day 90 in Period F-IV (HRT ¼ 2 d) (FIG. 9). The highest lactate selectivity reached 86.2±0.1% on day 94 and 87.2±0.2% on day 120 before the performance of the fermentation reactor deteriorated between days 108-112 and 124-126 due to a dysfunction in the pH probe (pH >10) and the activity of lactate producing microbes was significantly suppressed. However, the lactate concentration recovered, reaching more than 200 mM C with a selectivity of more than 70% within ten days after adjusting the pH back to 5.0-5.5 (FIG. 4A). After lowering the HRT, the overall COD conversion efficiency slightly declined to reach 68.2±1.8% on day 90, which could be due to the washout of more biomass from the fermentation reactor at the start of Period F-IV (HRT ¼ 2 d). However, the COD conversion efficiency gradually recovered to 80.4±1.1% on day 134, and lactate accounted for 51.5±0.2% of the total COD conversion in Period F-IV (HRT ¼ 2 d). The hydrogen and CO₂ gas production during Period F-IV remained low (0.7 L CO₂ d⁻¹ and 0.4 L H₂ d⁻¹) due to homolactic fermentation. These results suggest that the stability of the fermentation reactors was not impacted significantly by increasing the organic loading rate by lowering the HRT.

To further improve the lactate production in the fermentation reactor, the organic loading was further increased using non-diluted feedstock in Period F-V (days 135-166). Lactate concentration immediately increased from 255.9±10.8 mM C on day 136-544.0±6.6 mM C on day 138. It continued to increase, reaching a maximum value of 937.8±18.9 mM C on day 166 in Period F-V (no dilution) (FIG. 4A), around 3.7-fold higher than day 136. The higher lactate concentration using non-diluted CM feedstock in this study was close to what was achieved by Xu et al. using acid whey as feedstock and could trigger higher MCCA production in the second-stage chain elongation process. A part from lactate concentration, the selectivity of lactate in the product profile was also improved in Period F-V, as only lactate and acetate were detected after day 142. The average lactate selectivity reached 87.6±5.9% during Period F-V (FIG. 10), reaching 96.2±0.3% on day 146, and was significantly higher compared to that during Period F-IV (t-test, p value <0.05). This lactate selectivity was comparable to or even higher than what has been reported with other studies on food waste fermentation, which was beneficial for the subsequent MCCA production in the chain elongation reactor. However, the average conversion efficiency of influent COD to VFAs and lactate was considerably lower at 47.9±6.2% in Period F-V. This might be attributed to the significant COD loss for the gas production (1.7 L CO₂ d⁻¹ and 0.9 L H₂ d⁻¹) and the higher amounts of solid residues in the fermentation effluent during Period F-V. Overall, the lactate selectivity and production from expired dairy/beverage waste could be significantly enhanced by increasing the temperature to 43° C. and the organic loading rate, which represented a feasible strategy to provide feed source and electron donors for higher MCCA production in the following chain elongation reactor.

Performance of Chain Elongation Reactor with Different Medium Composition as Feedstock

To identify the impacts of different feed sources and electron donors on MCCA production in the chain elongation reactor, chain elongation microbes enriched from the mixed sludge sources were first fed with a synthetic medium containing 800 mM C ethanol (EtOH) and 400 mM C acetate (Ac) in Period CE-I (FIG. 5A). Lactic acid (LA) was added to the synthetic medium in Period CE-II and its concentration gradually increased to reach a concentration of 1050 mM C in Period CE-V, while the concentrations of acetate and ethanol were gradually decreased to reach 0 mM C. Results showed that the concentration of acetate in the chain elongation effluent was 403.0±47.6 mM C during Period CE-I (EtOH 800, Ac 400 mM C) and CE-II (EtOH 800, Ac 400, LA 300 mM C) (FIG. 5B), suggesting it was not consumed by microbes. Acetate likely had a negligible

contribution to caproate production in the chain elongation reactor, as ethanol consumption during Period CE-I and ethanol/lactate during Period CE-II was only observed. Lactate, compared to ethanol, seemed to be favored by chain elongation microbes, as it was almost completely consumed in Period CE-II, while the consumption of ethanol significantly decreased from 646.3±33.3 mM C during Period CE-I to 488.9±47.0 mM C during Period CE-II (t-test, p value ¼ 0.0016). Lactate played an important role in the caproate production despite a reduction of acetate and ethanol concentrations in the feed during the whole experiment period (FIG. 5A), as the average caproate concentration in the chain elongation effluent increased significantly from 105.3±25.1 mM C in Period CE-II (EtOH 800, Ac 400, LA 300 mM C) to 613.2±149.8 mM C in Period CE-V (LA 1050 mM C) (FIG. 5B) (t-test, p value ¼ 0.0004). The highest butyrate concentration was observed on day 46 during Period CE-IV, reaching 764.8±8.0 mM C using a synthetic medium containing 0 mM C acetate, 400 mM C ethanol, and 1050 mM C lactate. The corresponding caproate concentration was 261.6±20.4 mM C on day 46 and increased to 761.4±30.0 mM C at the end of Period CE-V (LA 1050 mM C). The concentrations of butyrate and caproate in the effluent of the chain elongation reactor during Period CE-V slightly exceeded the theoretical amount of lactate conversion to butyrate and caproate (700 mM C) through reverse b-oxidation, which could be attributed to the remaining ethanol/acetate from Period CE-IV (FIG. 5B) and the yeast extract that was added into the synthetic medium.

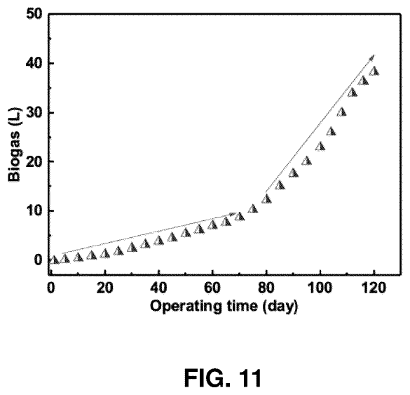

After gradual adaptation with a synthetic lactate-containing medium for ten weeks, the chain elongation reactor in Period CE-VI was only fed with unfiltered and unsterilized effluent generated from the fermentation reactor. The lactate concentration was measured at around 810-840 mM C in the fermentation reactor effluent (FIG. 5A), with a small amount of acetate (<50 mM C), solid residues, and non-digested COD. The lactate was completely consumed in the chain elongation reactor to mainly form caproate (524.5±24.7 mM C) and butyrate (338.8±20.9 mM C) (FIG. 5B), partially supporting the hypothesis above that lactate, compared to ethanol, seemed to be favored by chain elongation microbes in this study and the chain elongation reactor was not impacted by the complete removal of ethanol and acetate. The results from CE-V and CE-VI show that the caproate concentration and composition in the product profile was much higher with lactate as the sole substrate and electron donor than ethanol/acetate mixed feed in CE-I to CE-IV. The lactate-to-MCCA conversion ratio was higher than the theoretical value, possibly due to the microbial conversion of the remaining organics and residues in the fermentation reactor effluent. The biogas production (H₂: ˜ 81%, N₂: ˜3%, and CH₄: 16%) from the chain elongation reactor also increased with the increase in the concentration of lactate, reaching 24-40 L d⁻¹ in Periods CE-V and CE-VI (FIG. 11). The higher production of H₂ than CH 4 could be attributed to the acidic pH maintained in the reactor, which inhibits methanogens.

The maximum caproate concentration (14.8 g L⁻¹, 764.8±8.0 mM C) obtained with lactate as the sole electron donor in the chain elongation reactor was comparable to or even higher than most studies on lactate-driven chain elongation process as shown in Table 6. The temperature-phased two-stage fermentation process for specific organic waste valorization might generate higher lactate concentration in the lactate-fermentation process, which triggers the higher MCCA production in the following chain elongation process. This could be attributed to the high relative abundance of lactate-producing microbes after enrichment in the fermentation reactor, avoiding the competition between lactate-, butyrate- and caproate-producing microbes. The average caproate production rate in Period CE-VI reached 209.8±9.9 mM C d⁻¹ using the effluent from the fermentation reactor as feed for the chain elongation reactor (FIG. 5C), which was higher than what had been reported in similar studies using acid whey as the starting raw material.

TABLE 6
Comparison of caproate production vs lactate driven chain elongation

| | | No. | | | | Max | Max | |
|---|---|---|---|---|---|---|---|---|
| | Reactor | of | | Dominant | RA^{d} | concentration | production | |
| Substrate | type | days | pH | microbiome | (%) | (g L⁻¹) | (g L⁻¹ day⁻¹) | Ref. |
| Thin stillage | 1 L reactor | 42 | 5.4~5.7 | Megasphaera | 57 | 2.08 | 0.58 | [13] |
| + H2 | (with electro | | | elsdenii | | | | |
| | chemical cells) | | | | | | | |
| L - | 0.7 L | 220 | 5.0 | Acinetobacter | 62.9 | N/A^{c} | 3.03 | [14] |
| Lactate | anaerobic | | | sp. | | | | |
| | filter | | | | | | | |
| Grass | 100 ml reactor | 30 | 5.5~6.2 | Clostridium | 28 | 4.09 | N/A | [15] |
| | | | | IV | | | | |
| Acid | 0.7 L CSTRª | 90 | 5.0 | Bacteroidales | 21.7 | N/A | 1.68 | [16] |
| whey | | | | | | | | |
| 15x | | | Initial | Clostridiales | 12.6 | | | |
| diluted | 6 L leach-bed | 100 | pH | | | 9.59 | N/A | [17] |
| food waste | | | 7.0 | Clostridium | 42 | | | |
| leachate + H2 | | | | | | | | |
| | | | | Ruminococcaceae | 20.5 | | | |
| Acid | 1 L UASB^{b} | 400 | 5.5 | | | 10.4 | 3.20 | [18] |
| whey | | | | | | | | |
| | | | | Prevotellaceae | 17.9 | | | |
| Liquor | 5 L anaerobic | 485 | 6.5 | Clostridium V | 25.5 | 3.59 | 10.9 | [19] |
| brewing | filter | | | | | | | |
| wastewater | | | | | | | | |
| Xylan + | 1 L CSTR | 148 | 5.5 | Ruminiclostridium 5 | 42.3 | N/A | 3.60 | [20] |
| lactate | | | | | | | | |
| 10-15% | 1 L stirred | 45 | 6.0 | Caproiciproducens | 32-72 | 5.4 | 1.81 | [21] |
| (vol/vol) | tank reactor | | | | | | | |
| food waste | | | | | | | | |
| Lactate | 0.9 L CSTR | 194 | 5.0-6.5 | Caproiciproducens | 2-67.8 | 8.58 | N/A | [22] |

^{a}CSTR, continuously stirred tank reactor.
^{b}UA SB, upflow anaerobic sludge blanket.
^{c}N/A, data not available.
^{d}RA, relative abundance of the dominant caproate-producing microbial species.

The relatively high selectivity of caproate (˜85%) calculated by dividing the concentration of electrons in the formed caproate by the net consumed electrons from lactate in Period CE-VI was comparable to previous studies, leading to an 8.6-fold higher extraction rate for caproate (256±15 vs. 30±5 mmol C m² d⁻¹ for butyrate) from the chain elongation reactor using a continuous membrane-liquid extraction unit. This could further enhance the selectivity of longer-chain fatty acids after chain elongation, which could later reduce the cost of MCCA purification. Considering a relatively low membrane area-to reactor volume of 0.025 m² L⁻¹, a low extraction efficiency of 3% was achieved in this study, which is expected to be further improved by increasing the membrane area-to-reactor volume to 0.5 m² L⁻¹. The results of this study demonstrated the feasibility of the two-stage lactate-driven chain elongation process for the successful valorization of expired dairy and beverage waste.

### Microbial Communities and Co-Occurrence Analysis

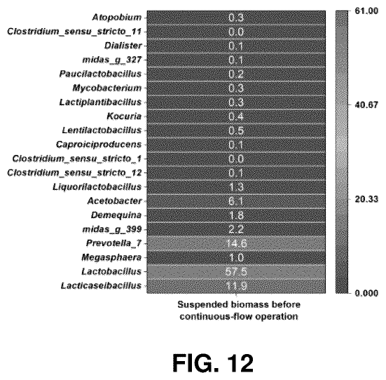

Dominance of lactate-producing genera in the fermentation reactor The suspended biomass in the fermentation reactor before switching to continuous-flow operation was mostly dominated by the genus Lactobacillus (57.5%), followed by Prevotella 7 (14.6%) and Lacticaseibacillus (11.9%) (FIG. 12). Lactobacillus and Lacticaseibacillus are known for their capability of converting sugars and other organics into lactic acid, while Prevotella 7 has been reported to be correlated with lipid and carbohydrate metabolism to produce odd-chain fatty acid (OCFA). For the lactate mediated chain elongation process, using dairy waste as the initial feed and inoculum source might be a good strategy for the fast enrichment of lactate-producing microbes in the fermentation reactor.

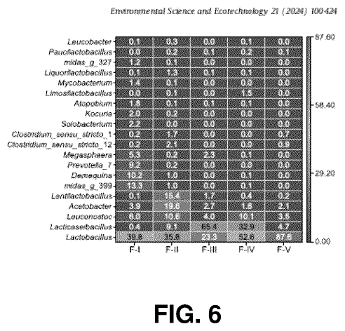

A heatmap of the top 20 OTUs in the suspended biomass of the fermentation reactor displayed the enrichment and dominance of lactate-producing microbes (Lactobacillus, Lacticaseibacillus, and Leuconostoc) under different conditions (FIG. 6). The combined relative abundance of lactate-producing genera exhibited a significant improvement from 46.2% (30° C., Period F-I) to 92.7% (43° C., Period F-III), resulting in the generation of lactate as the major fermentation product at 43° C. (FIG. 4A). In contrast, the relative abundance of OCFA producers (i.e., Lentilactobacillus and Prevotella 7:9.2-0%) and butyrate fermenters (Megasphaera:

5.3-2.3%, and Solobacterium: 2.2-0%) decreased with the temperature increase. The genus Demequina, capable of decomposing carbohydrates, also saw a reduction in its relative abundance from 10.2% to 0% with the temperature increase.

Collectively, these results show that operating the fermentation reactor at a high temperature resulted in the dominance of lactate producing microbes, which enhanced the overall conversion and utilization of carbohydrates into lactate (FIG. 4A). This was demonstrated by the significantly higher average lactate concentration (187.9±49.0 mM C) and selectivity (63.8±9.7%) at 43° C. (Period FIII) compared to 30° C. (almost 0 mM C, 0.7±1.5%, Period F-I) (t test, p<0.01) (FIG. 4A). Increasing the organic loading rate further enhanced the dominance of lactate-producing microbes, with the combined relative abundance of Lactobacillus, Lacticaseibacillus, and Leuconostoc increasing from 92.7% (Period F-III, HRT of 4 days) to 95.6% (Period F-IV, HRT of 2 days) and 95.8% (Period F-V, CM with no dilution) (FIG. 6). The higher organic loading rate seems to favor the enrichment of Lactobacillus over Lacticaseibacillus as the relative abundance of Lactobacillus increased from 23.3% (Period F-III) to 52.6% (Period F-IV) and further to 87.6% (Period F-V), whereas the relative abundance of Lacticaseibacillus decreased from 65.4% (Period F-III) to 32.9% (Period F-IV) and finally reached 4.7% (Period F-V). The high dominance of Lactobacillus with increasing organic loading rate contributed to the increase in the percentage of lactate-C in the whole product composition, from 61.9% on day 86 in Period F-III to 66.6% on day 134 in Period F-IV and 95.7% on day 166 in Period F-V. The enrichment level of lactate-producing microbes in this study was higher than previous studies using food waste for MCCA production but was comparable to a study with acid whey as feedstock in a two-stage chain elongation platform. This demonstrates the advantage of using dairy waste to enrich lactate-producing microbes in a two-stage chain elongation platform, which allows the separate optimization of operation conditions for the first-stage fermentation reactor to achieve satisfactory lactate production for the subsequent MCCA production.

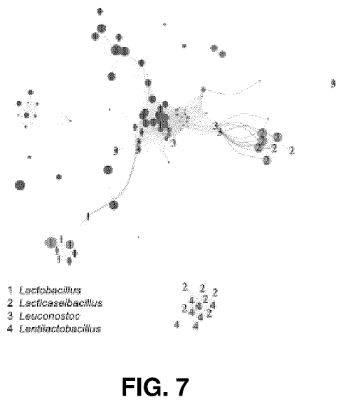

The potential symbiotic interactions between the different microbial OTUs in the fermentation reactor were analyzed via a cooccurrence network (FIG. 7). A total of 833 strong correlations were discovered for 103 microbial species, among which 98% were positively linked. The majority of the species in the most dominant genera (i.e., Lactobacillus and Lacticaseibacillus) were found to have positive correlations within their genus for lactate production. However, species from the genus Leuconostoc were found to be mostly positively linked with species from the genus Lactobacillus, indicating their potential symbiotic interactions. Besides, over 30 positive correlations were observed between species within the genera clusters of butyrate-producer Clostridium sensu stricto (red nodes), which explains the generation of butyrate in the fermentation reactor. However, further experiments are needed to confirm these interactions revealed by the co-occurrence network.

### Dominance of Chain Elongation Microbes Via Lactate Mediation

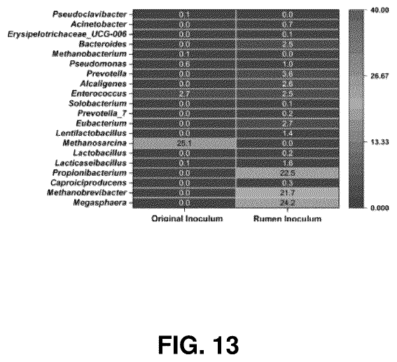

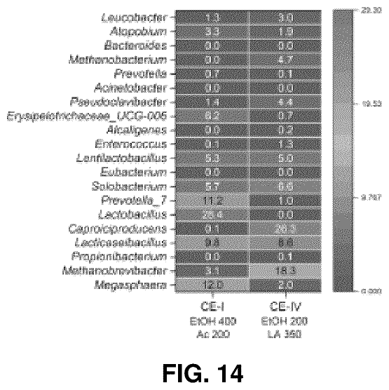

The initial anaerobic sludge inoculum used to seed the chain elongation reactor was mainly dominated by Methanosarcina (25.1%, FIG. 13). Lactobacillus, Prevotella 7, and Lacticasei bacillus became the dominant genera in Period CE-I (FIG. 14), mainly due to the introduction of sediments from the fermentation reactor. During this period, when ethanol and acetate (no lactate) were added to the reactor, the dominant chain elongator belonged to Megasphaera, but its relative abundance was relatively low (12.0%), which explains the low caproate production (FIG. 5B). Efficient chain elongation did not start until lactate was utilized as the carbon source and sheep rumen was added as inoculum source between Period CE-II and CE-IV (FIG. 5B). The microbial communities in the sheep rumen were dominated by chain elongating bacteria (FIG. 13 and 24.2% Megasphaera, 2.7% Eubacterium, 2.6% Alcaligenes), propionate producers (22.5% Propionibacterium) and methanogens (21.7% Methobrevibacter). After a series of adaption to lactate from Period CE-II (EtOH 800, Ac 400, LA 300 mM C) to CE-IV (EtOH 400, LA 1050 mM C) (FIG. 5A), the dominant chain elongating microbe shifted to Caproici producens (26.3%) in the suspended biomass at the end of Period CE-IV (FIG. 14), which resulted in the increase of MCCA proportion, especially caproate (FIG. 5B), in the whole product spectrum.

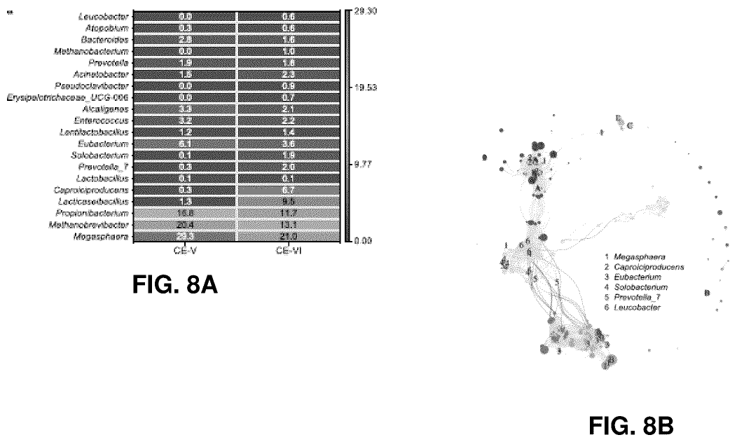

However, the operation of the microbial chain elongation reactor with lactate as the carbon source and mediator in Period V (LA 1050 mM C) shifted the dominance from Caproici producens to another chain-elongating bacteria, Megasphaera (29.3%, FIG. 8A), possibly because of the addition of sheep rumen, which had a high relative abundance of Megasphaera (24.2% Megasphaera, FIG. 13), and the complete elimination of ethanol. In a previous study, sheep or cow rumen was a common source for isolating Megasphaera spp. for MCCA production. Megasphaera hexanoica has been reported to be capable of generating caproate from lactate as a pure culture in previous studies. Further tests to feed the chain elongation reactor with the effluent from the fermenter, containing around 810-840 mM C lactate, did not change the dominance of chain elongating microbes (21.0% Megasphaera, 6.7% Caproici producens, and 3.6% Eubacterium) in Period CE-VI (fermentation effluent) compared to CE-V (LA 1050 mM C). The emergence of other chain elongators, such as Acinetobacter (2.3%) and Alcaligenes (2.1%), was detected in Period CE-VI (fermentation effluent).

These results suggest that the microbial community structure could be impacted and shaped by the change of substrates and inoculum sources, resulting in the dominance of chain elongators and higher production of MCCAs. The interactions between different genera in the chain elongation

reactor were illustrated by the co-occurrence network (FIG. 8B). A total of 239 species in the chain elongation reactor formed 2866 correlation connections between each other, among which 99% were positively linked with pink lines. The main chain elongation genera (Megasphaera, Caproici producens, and Eubacterium) and OCFA producers (Prevotella 7 and Leucobacter) were listed in the figure with node size proportional to their relative abundance. Over 85 positive correlations were found for the chain elongation species belonging to the genus Caproici producens within its own genus and with species from the genera Ruminococcus and CAG-352. Species belonging to the above three genera all belong to the family Ruminococcaceae, the correlations within which were not revealed previous studies. For the other caproic acid-producing bacteria, Megasphaera belongs to the family Veillonellaceae and was found to be positively correlated with species from the genera Bacteroides, whose members are commonly found in lactate-driven chain elongation microbiomes, and Solobacterium, a butyrate fermenter. These tight interconnections indicated their potential symbiotic partnership, contributing to the final high concentration of MCCAS in Period CE-VI (fermentation effluent). Twenty-three negative correlations were found between the chain elongating bacteria Alcaligenes, OCFA producer Prevotella 7, and lactate-producing species Lacticaseibacillus. Chain elongation species from the genus Alcaligenes may compete for lactate with Prevotella 7 and Lacticaseibacillus in the chain elongation reactor.

### Conclusions

The feasibility and advantage of lactate-mediated chain elongation to produce MCCAs was demonstrated here using expired dairy and beverage waste. The fermentation reactor operated at 43° C. was highly enriched with lactate-producing microbes, mainly Lactobacillus and Lacticaseibacillus, with a combined relative abundance of more than 90%, which promoted the high concentration and selectivity of lactate (81-96% based on mM C) in the product spectrum through the positive correlations between different OTUs. Lactate, as the sole mediator and carbon source for chain elongation, was efficient for producing MCCAs with microbial community structure dominated by the chain elongation microbes Megasphaera and Caproici producens. The study provided proof of concept for the valorization of dairy and beverage waste using a two-stage lactate-mediated chain elongation platform.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1. A bioreactor comprising:
a shell defined by one or more walls and a length, and
a plurality of hollow fiber membranes inside the shell,
wherein between about 20% and about 50%, between about 20% and about 30%, or about 50% of the length of the shell remains unoccupied by the plurality of porous hollow fiber membranes.

2. The bioreactor of claim 1, wherein: (a) one end of the plurality of porous hollow fiber membranes is mounted at a first end of the shell and the other end of the plurality of porous hollow fiber membranes is mounted at a second portion of the shell or (b) one end of the plurality of porous fiber membranes is mounted at a first end of the shell and the other end of the plurality of porous hollow fiber membranes is mounted at about the middle of the shell.

3. The bioreactor of claim 1, wherein the plurality of porous hollow fiber membranes comprises polymeric materials, non-polymeric materials, or a combination thereof.

4. The bioreactor of claim 1, wherein porous hollow fiber membranes in the plurality of porous hollow fiber membranes (a) comprise cellulose, cellulose acetate, polysulfone, polyacrylonitrile, inorganic carbon, alumina, polypropylene, polyethylene, polyvinylidene fluoride, polytetrafluoroethylene, polyether sulfone, sulfonated polyether sulfone, or a combination thereof; (b) are potted at both ends with a material selected from polyepoxides (such as solvent-resistant polyepoxides), polyurethane, polypropylene, or a combination thereof; (c) are configured as cylindrical tube bundles, helically wound bundles, rectangular bed of fibers, or a combination thereof.

5. The bioreactor of claim 1, wherein (a) the shell has a shape selected from a cylinder, rectangle, square, pentagon, hexagon, or octagon, or a combination thereof or (b) the shell comprises a material selected from polypropylene, polyvinylidene fluoride, polyvinyl chloride, metals (such as silver, zinc, copper, aluminum, nickel, iron, titanium, and chromium), metal alloys of any of the preceding metals, ceramics, glass, borosilicate-tempered glass, steel, plastics, ceramics, composites, quartz, silicon, or a combination thereof.

6. The bioreactor of claim 1, wherein the bioreactor comprises biocarriers in the shell volume, wherein the biocarriers are selected from the group consisting of/are made from a material selected from the group consisting of granular activated carbon, glass, polystyrene beads, plastic, polypropylene, polyethylene, polyvinyl dichloride, polytetrafluoroethylene, latex, rubber, agarose, or a combination thereof.

7. The bioreactor of claim 1, comprising microorganisms, wherein the microorganisms are sequestered on the biocarriers, within pore spaces of the biocarriers, or a combination thereof, and optionally, wherein the microorganisms comprise active chain-elongation organisms.

8. A method of extracting one or more compounds from a broth, the method comprising:
contacting a shell side stream containing the broth with the plurality of porous hollow fiber membranes of the bioreactor of claim 1.

9. The method of claim 8, wherein: (a) a solvent flows axially through the plurality of porous hollow fiber membranes; (b) the shell side stream and solvent flowing axially through the plurality of porous hollow fiber membranes flow in a co-current pattern, a counter-current pattern, or a cross-current pattern, or a combination thereof; and/or (c) wherein the solvent flowing axially through the plurality of porous hollow fiber membranes comprises mineral oil solvent with tri-n-octylphosphine oxide (e.g., mineral oil solvent with 3% tri-n-octylphosphineoxide), N-methylpyrrolidone, methyl isobutyl ketone, xylene, n-butanol, 1,2-butanediol, or a combination thereof.

10. The method of claim 9, the method comprising:
contacting the solvent that flows axially through the plurality of porous hollow fiber membranes with a pertraction solution after the solvent exits the plurality of porous hollow fiber membranes, wherein: (a) the pertraction solution has an alkaline pH, such as between 8 and 14, between 9 and 13, or between 9 and 11; (b) the bioreactor is maintained at a temperature between 28° C. and 35° C.; (c) the shell side stream containing the broth is maintained at a pH between 5 and 6, such as 5.5; and/or (d) the method comprising recirculating biogas through the bioreactor.

11. The method of claim 9, wherein:
(i) the pH of the bioreactor broth is maintained at 5.5,
(ii) the bioreactor has a hydraulic retention time of about one day, and
(iii) biogas is recirculated every 2 hrs for 5 mins, at a rate of 150 mL/min.

12. The method of claim 11, wherein the one or more compounds are medium chain carboxylic acids.

13. The method of claim 9, wherein the broth comprises a lactate solution comprising lactate at a concentration ranging between 800 and 2000 mM C, between 800 and 1800 mM C, between 800 and 1600 mM C, between 800 and 1400 mM C, between 800 and 1200 mM C, or between 800 and 1000 mM C at 43° C.

14. The method of claim 13, wherein the method of preparing the broth comprises inoculation and acclimatization of a fermentation bioreactor in a batch mode operation step, followed by a continuous mode operation step.

15. The method of claim 14, wherein the continuous mode operation step comprises feeding pre-treated biomass feedstock to the fermentation bioreactor, wherein the treated biomass is heated between 90° C. and 110° C., between 95° C. and 105° C., between 98° C. and 102° C., or between 99° C. and 101° C., and centrifuged between 3500×g and 4500×g, between 3700×g and 4300×g, or between 3900×g and 4100×g.

16. The method of claim 13, wherein the continuous mode operation step is conducted with a temperature between 25 and 55° C., between 25 and 50° C., between 25 and 45° C. or between 3° and 45° C.

17. The method of claim 13, wherein the continuous mode operation step is conducted with a Hydraulic Retention Time (HRT) between 1 and 6 d, between 1 and 5 d, between 1 and 4 d, between 1 and 3 d, or between 1 and 2 d.

18. The method of claim 13, wherein the continuous mode operation step is conducted with an organic loading rate between 1.0 and 40.0 g COD L⁻¹ d⁻¹, between 1.0 and 35.0 g COD L⁻¹ d⁻¹, between 1.0 and 30.0 g COD L⁻¹ d⁻¹, between 2.0 and 30.0 g COD L⁻¹ d⁻¹, between 3.0 and 30.0 g COD L⁻¹ d⁻¹, or between 4.0 and 30.0 g COD L⁻¹ d⁻¹.

19. The method of claim 13 wherein the continuous mode operation step is conducted in five separate periods.

20. The method of claim 14, wherein the acclimatization comprises feeding organic mass; optionally expired milk, yogurt, and fruit juice; mixed at a ratio based on the actual percentage of each expired organic mass in the total organic mass to the fermentation bioreactor.
